(19) United States
(12) Patent Application Publication
(10) Pub. No.: US 2010/0316609 A1
(43) Pub. Date: Dec. 16, 2010

(54) Conditionally Replicating Viruses for Cancer Therapy

(75) Inventors: Stephen Dewhurst, Rochester, NY (US); Baek Kim, Rochester, NY (US)

Correspondence Address: McKeon Meunier Carlin & Curfman LLC, 817 W. Peachtree Street, Suite 900, Atlanta, GA, 30308, US

(73) Assignee: UNIVERSITY OF ROCHESTER, Rochester, NY (US)

(21) Appl. No.: 12/446,136

(22) PCT Filed: Oct. 18, 2007
(86) PCT No.: PCT/US07/81760

§ 371 Date: Sep. 28, 2009

Related U.S. Application Data
(60) Provisional application No. 60/829,983, filed on Oct. 18, 2006.

(51) Int. Cl. A61K 48/00; C12N 15/85; A61K 35/00; A61K 35/76; A61K 31/70; A61K 31/713; A61P 35/00

(52) U.S. Cl. 424/93.2; 435/320.1; 514/44 R

(57) ABSTRACT

Described herein are viral vectors comprising a nucleic acid encoding a viral DNA polymerase wherein the encoded viral DNA polymerase comprises at least one amino acid modification. Also provided are methods of making and using the viral vectors.

### STATEMENT REGARDING FEDERALLY FUNDED RESEARCH

The invention was made with government support from the National Institutes of Health, Grant No. R01 AI49781. The government may have certain rights in this invention.

### BACKGROUND

Many human cancers are associated with a very poor prognosis. There is thus considerable interest in the development of novel therapeutic approaches for cancer. At the genetic level, cancers contain alterations in various genes that regulate cell proliferation, including tumor suppressors, proto-oncogenes and other cell growth controlling genes. In some cases, these genetic mutations represent potential targets for anti-cancer gene therapies, which may seek either (i) to functionally correct the altered genes or (ii) to reduce expression of growth-promoting genes using dominant negative mutant or RNAi-based approaches. Other approaches to cancer gene therapy include the introduction of genes that will induce apoptosis in the transduced tumor cell targets and the use of prodrugs/suicide genes, such as herpes simplex virus thymidine kinase (HSV TK) and bacterial cytosine deaminase (CD).

An additional approach is the use of selectively replicating, oncolytic viruses. This approach typically involves recombinant adenovirus and herpes simplex virus type-1 (HSV-1) vectors with specific mutations that allow them to replicate efficiently in tumor cells but not in normal cells. For example, modified adenovirus vectors that contained a deletion of the E1B region have been used. Viruses of this type (such as ONYX-015) replicate and induce lysis only in cancer cells that lack p53 function. Introduction of these oncolytic, conditionally-replicating adenoviruses (CRAD) to p53 defective cancers have shown generally promising initial results in phase I and II trials. In the setting of prostate cancer, an initial Phase I trial of ONYX-015 showed that the oncolytic CRAD was well-tolerated, but no objective responses were observed. A follow-up phase I/II trial used endoscopic ultrasound to deliver ONYX-015 directly to the primary tumor mass. This was found to be generally well tolerated, either alone or when combined with chemotherapy (gemcitabine), and, in some patients, partial regressions or stabilization of disease were observed. Collectively, these data suggest that oncolytic CRADs are well tolerated in the context of cancer. However, oncolytic CRADs often contain a deletion of E1B, which results in virus replication only in p53-deficient tumor cells, or a mutation in the E1A locus that results in E1A expression only in the environment of a tumor cell (for example, by placing E1A under the transcriptional control of the telomerase reverse transcriptase [TERT] promoter). These approaches are, however, limited in that many tumors cells continue to express p53, while some normal cells may express TERT.

### SUMMARY

Described herein are viral vectors and compositions comprising a nucleic acid encoding a viral DNA polymerase wherein the encoded viral DNA polymerase comprises at least one amino acid modification. Also provided are methods of making and using the viral vectors.

The details of one or more embodiments are set forth in the accompanying drawings and the description below. Other features, objects, and advantages will be apparent from the description and drawings, and from the claims.

### DESCRIPTION OF DRAWINGS

FIG. 1 shows dNTP concentration dependent RT activity of wild type and mutant HIV-1 RTs. A ³²P labeled 17mer primer (S) annealed to a 38mer RNA template was extended by three HIV-1 RT proteins (A, B and C) with different dNTP concentrations ([dNTP]), (lanes 1-8). Lane 5=0.25 μM. A/T, R/T and M indicate [dNTP] found in human activated T cells, resting T cells and macrophages.

FIG. 2 shows HIV-1 RT based dNTP assay for TTP between 4 and 128 fmole in 18/19mer T/P (A) and TTP standard curve (B). A ³²P labeled 18-mer annealed to a 19-mer template was extended by HIV-1 RT with known amounts of TTP. Percent of the primer extension with each dNTP amount was plotted (B).

FIG. 3 shows transduction of human CD4+ T cells (A) or macrophages (B, C) by wildtype and mutant HIV-1 vectors expressing GFP. The GFP expression was analyzed by FACS at 48 hours post transduction with an equal MOI of wild type, V148I and Q151N HIV-1 vectors.

FIG. 4 shows the effect of exogenous deoxynucleosides (dNs) on the transduction of human lung fibroblasts by wild type and Q151N HIV-1 vectors. FIG. 4A shows transduction of cells in 10% FBS (bright field and matched fluorescence image showing GFP). FIG. 4B shows transduction of cells pretreated with exogenous dNs.

FIG. 5 shows valproic acid (VPA) enhances gene expression from a replication-defective adenovirus type 5 vector in vitro. FIG. 5A is a graph showing N2A cells transduced by Ad.CMV-LacZ, and then exposed to varying concentrations of VPA (as indicated). Twenty-four hours later, LacZ expression was analyzed. FIG. 5B is a graph showing N2A cells cultured on poly-D-lysine coated 12-well plates in NB/B27 medium, and transduced with Ad.CMV-LacZ at a multiplicity of infection (MOI) of 10. Two hours thereafter, the culture medium was replaced with medium containing 1 mM VPA or vehicle (control). The medium was changed every 24-hours thereafter, and cell aliquots were harvested at the indicated time points (1, 3, 5, 7, 10 and 12 days post infection). LacZ expression was then measured in the lysates.

FIG. 6 shows VPA enhances gene expression from a replication-defective adenovirus type 5 vector in vivo. In FIG. 6A luciferase-encoding adenovirus vector (Ad.CMV-Luc) was inoculated into the thigh muscle of BALB/c mice. These animals (3/group) were then treated with either VPA or with vehicle alone for six days, by twice-daily i.p. injection. Representative images obtained with the IVIS CCD camera at day 2 and 6 post Ad-infection are presented. In FIG. 6B the levels of adenovirally vectored luciferase gene expression in the mice shown in panel A were quantitated at day 2 and 6 post infection using the Living Image® 2.20.1 software (Xenogen, Alameda, Calif.).

FIG. 7 shows [dNTP]-dependent DNA polymerization assays with various HIV-1 RT polymerase mutants, and their wild-type counterpart (WT). A 5′ end ³²P end labeled DNA primer was annealed to a RNA template and extended by equivalent amounts of reverse transcriptase activity showing full extension of 50% labeled primers at 250 μM in a short reaction at 37° C. dNTP concentrations are denoted at the side of the Figure. The dark dots at the bottom of each lane represent the substrate, while the uppermost band in each panel represents the full-length extended product.

FIGS. 8A and 8B are bright field (8A) and fluorescence (8B) images of virus plaque in a 293 cell monolayer demonstrating that an adenovirus mutant containing the I664V polymerase mutation replicates in 293 cells in the presence of exogenous dNTPs (1 mM).

FIGS. 9A, 9B and 9C are [dNTP]-dependent DNA polymerization assays with Pfu DNA polymerase mutants with mutations in residues 408, 409 and 410, respectively, and their wild-type counterpart (WT). A 5′ end ³²P-end labeled DNA primer was annealed to a DNA template and extended by equivalent amounts of DNA polymerase activity showing full extension of 50% labeled primers at 250 μM in a short reaction at 37° C. dNTP concentrations are denoted below the Figure. S denotes the substrate, F denotes the full-length extended product.

### DETAILED DESCRIPTION

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting. Note the headings used herein are for organizational purposes only and are not meant to limit the description provided herein or the claims attached hereto.

Oncolytic viral vectors are described that comprise a mutant viral DNA polymerase active only at high dNTP concentrations. Methods for the generation of an oncolytic virus comprising mutant viral DNA polymerases that are active only at high dNTP concentrations are also described. Viral vectors which contain a mutant viral DNA polymerase active only at high dNTP concentration replicate selectively in tumor cells. As described below in the Examples, lentiviral vectors which contain mutated derivatives of the viral DNA polymerase (reverse transcriptase) that are active only at high dNTP concentrations, selectively transduce tumor cells (including PC cell lines) but not normal cells. This is because of the much higher proliferative rate and dNTP content of cancer cells as compared to normal cells.

Also provided are methods for generating oncolytic CRAD comprising a polymerase-mutant with other genetic mutations (including mutation(s) affecting expression of the E1 or E3 loci), which may result in CRADs that have improved safety and efficacy. In addition, vectors comprising a nucleic acid encoding a therapeutic gene are described.

Methods for treating cancer in a subject comprising administration of the provided oncolytic viruses are also described. The method optionally includes administration of a second therapeutic agent.

Viral DNA Polymerases

As used herein, DNA polymerase refers to a viral DNA polymerase. Viral DNA polymerases include, but are not limited to, adenoviral DNA polymerase, herpes simplex virus type-1 DNA polymerase, herpes simplex virus type-2 DNA polymerase, poxvirus DNA polymerase, lentiviral DNA polymerase, spumavirus DNA polymerase, and cytomegalovirus (CMV) DNA polymerase. Poxvirus DNA polymerases include vaccinia virus, modified vaccinia virus and nonhuman poxvirus DNA polymerases. Lentiviral DNA polymerases include human and nonhuman immunodeficiency virus DNA polymerases. Spumavirus or foamy virus DNA polymerases include human and nonhuman spumavirus DNA polymerases. CMV DNA polymerases include human and nonhuman cytomegalovirus DNA polymerases.

Preferably, the adenoviral DNA polymerase is adenovirus type 5 DNA polymerase (GenBank Accession No. AAW65499; see also AC_{—}000008 at http://www.ncbi.nlm.nih.gov/entrez/viewer.fcgi??db=nucleotide&val=56160529) or adenovirus type 19 DNA polymerase (GenBank Accession No. AAW32973). Preferably, the human lentiviral DNA polymerase is HIV-1 DNA polymerase, reverse transcriptase (see NC_{—}001802). Preferably, the simian lentivirus DNA polymerase is simian immunodeficiency virus reverse transcriptase (GenBank Accession No. AAL59620). For the HSV-1 polymerase, see NC_{—}001806 at http://www.ncbi.nlm.nih.gov/entrez/viewer.fcgi??db=nucleotide&val=NC_{—}001806. For HSV-2 polymerase see NC_{—}001798 at http://www.ncbi.nlm.nih.gov/entrez/viewer.fcgi??db=nucleotide&val=NC_{—}001798. For human cytomegalovirus (HCMV) polymerase see NC_{—}006273, for vaccinia (pox) virus polymerase see NC_{—}006998; for spumavirus polymerase (reverse transcriptase, RT) see NC_{—}001795.

As described herein the viral DNA polymerase comprises one or more amino acid modifications. Suitable modifications for adenoviral DNA polymerases include, but are not limited to, amino acid substitutions selected from the group consisting of I664S, GG666/7AA, Y690A, Y690F and K837A. Suitable modifications for the HIV-1 reverse transcriptase include, but are not limited to, amino acid substitutions selected from the group consisting of Q151N, and V148I, A114V, A114L, A114S, Y115F and Y115A. Suitable modifications for the simian immunodeficiency virus reverse transcriptase include, but are not limited to, amino acid substitution VI 481.

Provided are also nucleic acids encoding viral DNA polymerases comprising a nucleotide sequence at least about 70%, 75%, 80%, 85%, 86%, 90%, 95%, 98%, 99% or more identical to the nucleotide sequence found at GenBank Accession Nos. AAW65499, AAW32973 or AAL59620 or to a complement thereof. Provided are also nucleic acids that hybridize under stringent hybridization conditions to a nucleic acid molecule found at GenBank Accession Nos. AAW65499, AAW32973 or AAL59620 or to a complement thereof. Further provided are nucleic acids encoding viral DNA polymerases comprising nucleotide sequences with 70-99% identity with other nucleotide sequences provided herein, nucleic acids that hybridize under stringent conditions to a nucleic acid molecules provided herein.

The polypeptides described herein can be modified and varied so long as the desired function is maintained. As used herein, the terms polypeptide, protein and peptide are used interchangeably to refer to amino acid sequences. It is understood that one way to define variants and derivatives, of the disclosed genes and proteins herein is through defining the variants and derivatives in terms of homology to specific known sequences. For example GenBank Accession No. AAW65499 sets forth a particular amino acid sequence of the polypeptide encoded by a nucleic acid sequence. Specifically disclosed are variants of these and other genes and proteins herein disclosed which have at least about, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by inspection, BLAST and BLAST 2.0 and algorithms described by Altschul et al., Nucleic Acids Res. 25:3389-3402, 1977; Altschul, et al., J. Mol. Biol. 215:403-410, 1990; Zuker, M. Science 244:48-52, 1989; Jaeger et al. PNAS USA 86:7706-7710, 1989 and Jaeger et al. Methods Enzymol. 183:281-306, 1989. Each of these references is incorporated by reference at least for the material related to alignment and calculation of homology.

The same types of homology can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

It is understood that any of the methods typically can be used and that in certain instances the results of these various methods may differ. Where sequence similarity is provided as, for example, 95%, then such similarity must be detectable with at least one of the accepted methods of calculation.

Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Fusion protein derivatives are made by fusing a polypeptide to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Substitutions, deletions, insertions or any combination thereof may be combined. Substitutional variants are those in which at least one residue has been removed and a different residue inserted in its place.

As used herein, modification with reference to a polynucleotide or polypeptide, refers to a naturally-occurring, synthetic, recombinant, or chemical change or difference to the primary, secondary, or tertiary structure of a polynucleotide or polypeptide, as compared to a reference polynucleotide or polypeptide, respectively (e.g., as compared to a wild-type polynucleotide or polypeptide). Polynucleotides and polypeptides having such modifications can be isolated or generated using methods well known in the art.

The nucleic acids that can encode those protein sequences, variants and fragments thereof are also disclosed. These include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

DNA polymerase mutants with reduced activity at low dNTP concentrations are described. Sequence analysis of bacterial, viral and cellular DNA polymerases has led to the development of a classification system that includes four groups of enzymes: A, B (aka α-like), C and D. Adenovirus DNA polymerase (Ad Pol) falls into the second group and is therefore similar to human DNA polymerase-α as well as to the well-characterized polymerases encoded by RB69 and Phi29 bacteriophage. This family of polymerases contains six highly conserved motifs within the polymerase active site. Of these, three motifs are conserved in all groups of DNA polymerases, and residues within these motifs play crucial roles in dNTP or template/primer binding, or in enzyme catalytic activity. Residues within, or close to, two of these motifs (the A and B motifs) have been implicated in dNTP binding by the Phi29, and RB69 DNA polymerases (Table 1).

TABLE 1
Conservation of Ad DNA polymerase residues in the A and B motifs
Alignment of the A motif, and a “pre-A motif” ([I/Y]xGG (SEQ ID NO: 1)).

| | |
|---|---|
| RB69/387 | IxGG (SEQ ID NO: 2) Motif A |
| | PVQPYPGAFV-------KEPIPNRYKYVMSF [symbol] LTSL [symbol] PSI (SEQ ID NO: 3) |
| Pfu DNA | IxGG (SEQ ID NO: 2) Motif A |
| poly/381 | KRESYTGGFV---------KEPEKGLWENIVYLDFRALYPSI (SEQ ID NO: 4) |
| Phi29/222 | IxGG (SEQ ID NO: 2) Motif A |
| | VRYAYRGGFTWLNDRFKEKEIGEGMVF [symbol] VNSL [symbol] PAQ (SEQ ID NO: 5) |
| Ad5/660 | IxGG (SEQ ID NO:2) Motif A |
| | VRASIRGGRCYPTYLGILREP-LYVYDICGM [symbol] ASA (SEQ ID NO: 6) |

Underlined conserved residue Y416 in RB69 binds to the OH group on the ribose
moiety of the dNTP.
The other conserved residues are in bold font and include the [I/Y]xGG
(SEQ ID NO: 1) motif
Motif A is shown in italics.

Alignment of the B motif, and a “pre-B motif.”

| | |
|---|---|
| RB69/474 | Pre-Motif B Motif B |
| | EITKVFNQRKEHKGYMLA---(57)-RTEVAGMTAQINR [symbol] LLI [symbol] SL [symbol] GALG |
| | (SEQ ID NO: 7) |
| Phi29/359 | Pre-Motif B Motif B |
| | LFKDFIDKWTYIKTTS-----------------------EGAI-KQLA [symbol] LMLNSL [symbol] GKFA (SEQ ID NO: 8) |
| Ad5/809 | Pre-Motif B Motif B |
| | VAREYVQLNIAAKE-------------------ADRDKNQLTRSIA [symbol] LLSNAL [symbol] GSFA (SEQ ID NO: 9) |

The conserved lysines, known to directly bind dNTP in the Phi29 polymerase (K371,
K379 and K383), as well as their counterparts in the Ad5 DNA polymerase and in the
RB69 enzyme (K486 and K560) are underlined.
Other highly conserved residues are shown in bold font.
The B motif is shown in italics.

In the RB69 polymerase, biochemical and crystallographic studies have shown that highly conserved residues within, or close to, the B motif interact with the incoming dNTP, including Lys560 and Asn564 (which contact alpha and beta phosphate oxygens), and Arg482 and Lys486 (which contact gamma phosphate oxygens). In addition, residue Tyr416 (which is located in the A motif) interacts with the ribose moiety of the dNTP substrate, in a manner analogous to Tyr115 in HIV-1 RT. In the case of Phi29 DNA polymerase, which has not been crystallized, residues that are involved in direct dNTP binding include highly conserved lysines at positions 371, 379 and 383 all of which are located at, or close to, the B motif. The “pre-B motif” located close to the B motif has therefore been proposed to be potentially important for dNTP binding.

The DNA polymerase encoded by adenovirus type 5 contains highly conserved residues within motifs A and B (Table 1), several of which have been subjected to mutagenesis and subsequent functional analysis. Within the A motif, the equivalent residue to Y416 from RB69 DNA polymerase is Y690 in the Ad5 DNA pol. Substitution of this residue by alanine results in a polymerase that remains active, albeit at a reduced level (10% of wildtype) (Liu, et al., J. Virol. 74:11681-9 (2000)). Mutagenic analysis of a conserved “pre-A motif” ([I/Y]xGG (SEQ ID NO:1)) has revealed that this region has a significant, but indirect, effect on dNTP binding. This pre-A motif is believed to function as a DNA binding entity, based on the location of the corresponding residues in the crystal structure of RB69 bacteriophage DNA polymerase. In the RB69 DNA polymerase, the tyrosine in the (I/Y)XGG (SEQ ID NO:1) motif (Y391, corresponding to I664 in Ad Pol) interacts directly with the DNA template, approximately 12 angstroms away from the dNTP binding site. In the case of the Ad Pol, mutations in this region (I664S and GG666/7AA) resulted in impairment of DNA polymerase activity at low dNTP concentrations, and wild-type activity at higher dNTP concentrations. Indeed, in a primer extension assay, the I664S and GG666/7AA Ad Pol mutants required, respectively, 5× or 200× higher levels of dNTPs in order to carry out DNA synthesis (625 nM or 25 μM for 11664S or GG666/7AA, respectively, versus 125 nM for wild-type Ad Pol). Thus, these two Ad Pol mutants showed very similar enzymatic alterations to the V 1481 and Q151N mutants of HIV-1 DNA polymerase (RT). At a mechanistic level, these alterations in dNTP requirement for the two mutant Ad Pol enzymes can be explained on the basis that the mutations (particularly GG666/7AA) result in local destabilization of the template-primer complex at the polymerase active site. This in turn results in an alteration of the positioning of the template strand, which causes it to become incorrectly positioned with respect to base pairing to the incoming dNTP. The overall effect is to alter the affinity of the mutant polymerases for dNTP substrates (by almost 200-fold in the case of the GG666/7AA mutant).

Motif B from the adenovirus polymerase also contains a number of residues that affect dNTP binding. One mutant that has been examined, however, was an 837K/A mutant (a residue positionally equivalent to K383 in Phi29 DNA Pol, and K560 in RB69 DNA Pol). The Ad Pol 837K/A mutant had essentially normal (wild-type) polymerase, DNA binding and initiation activities, but its dNTP binding affinity was not tested (Liu, et al., J. Virol. 74:11681-9 (2000)). K560A mutant of the RB69 DNA polymerase showed greatly impaired catalytic activity (Yang, et al., Biochemistry 41:2526-34 (2002)).

Additional mutations that alter the dNTP binding properties of Phi29 or RB69 DNA polymerases are shown in Table 2 (Blasco M A et. al. J Biol. Chem. 268:16763-70, 1993; Truniger V. et. al. J Mol. Biol. 335:481-94, 2004; Truniger V. et. al. J Mol. Biol. 286:57-69, 1999; Yang G. et. al. Biochemistry 38:8094-101, 1999; Jacewicz A. et. al. J Mol. Biol. 368:18-29, 2007). The S392 residue in the Ad5 polymerase may also exert an effect on dNTP binding, since it is located in close proximity to the Y390 residue and contributes to DNA/template binding (Liu H. et. al. J. Virol. 74:11681-9, 2004).

TABLE 2
Adenovirus 5 DNA Polymerase Mutants With Reduced
dNTP Binding Affinity and/or Increased dNTP Concentration
Requirement for Efficient Activity

| | |
|---|---|
| Wild-type residues | Mutant |

| Phi 29 | RB69 | Ad5 | Ad5 Mutant |
|---|---|---|---|
| Y226 | Y391 | I664 | I664M |
| R227 | R335 | R665 | R665K |
| K379 | Q556 | R833 | R833K or R833T |
| K383 | K560 | K837 | K837A |
| N387 | N564 | N841 | N841Y or N841S |
| Y390 | Y567 | Y844 | Y844S |
| K392 | A569 | S692 | S692A |
| Y454 | Y619 | Y1010 | Y1010F |

Mutation of residues 408, 409 and 410 in the Pfu DNA polymerase from Pyrococcus furiosus, (GenBank Accession No. P61875) resulted in a reduction in polymerase activity at low dNTP concentrations. This reflects a drop in the dNTP binding activity of these mutants. As shown in Table 3, residues 408, 409 and 410 within the Pfu DNA polymerase correspond to conserved domains with the bacterioophage RB69 DNA polymerase and the adenovirus type 5 DNA polymerase.

TABLE 3
Conserved Residues Within Pfu, Rb69 and
Adenovirus Type 5 DNA Polymerases.

| Pfu residue | 408 | 409 | 410 |
|---|---|---|---|
| Pfu sequence | A | L | Y |
| RB69 sequence | S | L | Y |
| Adenovirus | G | M | Y |

As shown in the Examples below, mutation of residue 410 from Y (wild-type) to I (Y410I) resulted in a profound decline in DNA polymerase activity at low dNTP concentration. Y410L and Y410V mutations also result in a significant reduction in activity at low dNTP concentrations. Mutation of residue 409 from L (wild-type) to I (L409I) results in a significant drop in DNA polymerase activity at low dNTP concentration, as does the L409V mutation.

Mutations at the corresponding residues in the adenovirus DNA polymerase (which are located within Motif A of the polymerase) have a similar effect on the activity of this enzyme, based on sequence conservation.

Human immunodeficiency virus type-1 (HIV-1) is a lentivirus that encodes a DNA polymerase, reverse transcriptase (RT), that has the unique ability to utilize both RNA and DNA templates for the synthesis of a complementary DNA strand. The fundamental biochemical and structural properties of HIV-1 RT are surprisingly similar to those of other DNA polymerases. This is exemplified by the fact that HIV-1 RT can functionally substitute for DNA polymerase I in Escherichia coli. Moreover, the catalytic domains of the two enzymes contain identical or similar residues at common spatial positions, and a broad conversation of three-dimensional architecture.

Structural analysis of the HIV-1 reverse transcriptase (RT) and the ternary complex formed between the enzyme, its template/primer (T/P) and a deoxythymidine triphosphate (dTTP) substrate have revealed that several amino acid residues in the DNA polymerase active site of HIV-1 RT interact with the dNTP substrate. A comparison of the dNTP interaction residues of other DNA polymerases shows that in contrast to other DNA polymerases such as the Klenow fragment (KF) of E. coli DNA polymerase I, HIV-1 RT has a unique interaction with the 3′ OH at the sugar moiety of the incoming dNTP substrate. The side chain of the HIV-1 RT Q151 residue lies within 3.14 A of the 3′ OH of the dNTP substrate, suggesting that a hydrogen bond forms between these moieties. To test this unique interaction, the Q151N mutant HIV-1 RT was constructed and this mutant RT has reduced dNTP binding affinity (Kd) but unaltered enzyme catalysis (kpol). A mutation within the simian immunodeficiency virus (SIV) RT, V148I, was also identified, which displays kinetic alterations that are very similar to those shown by the HIV-1 Q151N RT mutant. Structural modeling predicts that the V148I mutation, which lies near to the Q151 residue, moves the side chain of the Q151 residue away from the active site, resulting in the loss of the 3′ OH interaction with the dNTP substrate; hence, the overall structural effect of the V148I mutation is analogous to that of the Q151N mutation. HIV-1 RT proteins containing these two dNTP binding mutations fail to synthesize DNA efficiently at low dNTP concentrations even though these RTs are fully active at high dNTP concentrations. Therefore, the Q151N and V148I HIV-1 RT mutants are functionally active only in the presence of high levels of dNTPs.

Mutation of the Y115 residue in HIV-1 reverse transcriptase, which corresponds to the conserved Y410 residue from the Pfu DNA polymerase, to a less bulky residue results in a dramatic decline in DNA polymerase activity at low dNTP concentrations, confirming the importance of this residue.

Since bacterial, viral and cellular DNA polymerases are well-characterized groups of enzymes: A, B (aka α-like), C and D. The conserved motifs within the polymerase active site can be exploited as described above to identify other mutations that result in DNA polymerases with activity only at high dNTP concentrations. This is true for all four classes of enzymes, A, B, C and D. Thus mutations described for one polymerase can be understood by one of skill in the art to be applicable to similar motifs in other polymerases.

One key biochemical difference between dividing and nondividing cells is their cellular dNTP concentration. Numerous studies have shown that dividing cells have higher dNTP concentrations than non-dividing cells, because a higher proportion of the cell population is in S phase where dNTP biosynthesis is activated (Traut, Mol. Cell Biochem. 140:1-22 (1994)). For the same reason, the dNTP content of cancer cells is much higher than normal cells.

As used herein the phrase normal dNTP concentration or low dNTP concentration refers to a range from about 0.05 μM to about 0.2 μM. As used herein the phrase high dNTP concentration refers to concentrations greater than and equal to about 0.25 μM. The dNTP concentrations of various cell lines are shown in Table 4. The mutant viral DNA polymerases described herein are active at high dNTP concentrations.

Described another way, the mutant viral DNA polymerase has from about 10 to about 100 fold lower activity at normal dNTP concentrations compared to its activity at high dNTP concentrations. Thus the mutant viral DNA polymerase can have 10 fold, 100 fold or any number in between 5 to 1000 fold lower activity at normal dNTP concentrations compared to its activity at high dNTP concentrations.

Ranges may be expressed herein as from about one particular value and/or to about another particular value. When such a range is expressed this includes the one particular value and/or the other particular value and any amount in between. It will be further understood that the endpoints of each of the ranges are significant both in relation to the other endpoint and independently of the other endpoint.

Enzymes involved in dNTP biosynthesis (e.g., ribonucleotide reductase, RNR) are upregulated when cells enter S phase, because dNTPs are an essential substrate for new DNA synthesis and chromosomal replication. Cellular dNTP availability can also be altered following exposure of cells to chemotherapeutic agents. For example, exposure of cells to hydroxyurea (HU, (Snyder, Biochem. Pharmacol. 33:1515-8 (1984))), which is an inhibitor of RNR, results in a reduction of cellular dNTP levels, whereas the treatment of cells with deoxynucleosides (dNs, precursors of dNTPs) increases cellular dNTP content (Fox, Pharmacol. Ther 30:31-42 (1985)).

A highly sensitive dNTP assay that has approximately 250-fold greater sensitivity than the standard, HPLC-based dNTP assay has been developed (Diamond, et al., J. Biol. Chem. 279:51545-51553 (2004)). This assay allows for determination of the cellular dNTP concentration in a range of primary cell types, including two primary human cell types that represent key targets for HIV-1 infection, terminally differentiated monocyte-derived macrophages and activated/dividing CD4+ T cells. Using this assay, human macrophages were shown to have about 50-100 times lower cellular dNTP concentration (˜50 nM) than activated CD4+ T cells (˜2-5 μM).

Based on the dNTP content of these target cells and the biochemical properties of the HIV-1 DNA polymerase, the efficiency of DNA synthesis by HIV-1 (and other retroviruses) is dependent on the availability of cellular dNTPs. Consistent with this, wild-type HIV-1 RT efficiently synthesizes DNA even at the low dNTP concentrations found in macrophages (˜50 nM), due to its tight dNTP binding affinity. In contrast, DNA polymerases from oncoretroviruses (which replicate only in actively dividing host cells) can synthesize DNA only at high dNTP concentrations, such as those found in tumor cells or actively proliferating cells (see the Examples below). Similarly, mutated derivatives of the HIV-1 and SIV DNA polymerases that have an altered ability to interact with cellular dNTP substrates (i.e., the HIV-1 Q151N mutant and the SIV V148I mutant described herein) can also synthesize DNA only at high dNTP concentrations (see the Examples below).

Vectors

Provided herein are viral vectors comprising a nucleic acid encoding a viral DNA polymerase wherein the encoded viral DNA polymerase comprises at least one amino acid modification. Suitable viral vectors include, but are not limited to, adenoviral vectors, herpes simplex virus type-1 vectors, herpes simplex virus type-2 vectors, poxvirus vectors including vaccinia virus, modified vaccinia virus and nonhuman poxvirus vectors, lentiviral vectors including human and nonhuman immununodeficiency viruses, spumavirus vectors including human and nonhuman spumaviruses and cytomegalovirus vectors including human and nonhuman cytomegaloviruses.

Adenoviral Vectors

The term adenovirus includes any of the 50+ human adenoviral serotypes currently known. Adenovirus and adenoviral vectors include any and all viruses that may be categorized as an adenovirus, including any adenovirus that infects a human or an animal, including all groups, subgroups, and serotypes. For example, adenovirus includes adenovirus type 3, 5, 7, 11, 19, 35 and 49 and non-human adenovirus such as a chimpanzee or avian adenovirus. However, the present application contemplates the use of all adenoviral serotypes to construct the oncolytic vectors and virus particles as described herein. For example, the adenoviral nucleic acid backbone can be derived from adenovirus serotype 2 (Ad2), 5 (Ad5), 19 (Ad19) or 35 (Ad35), although other serotype adenoviral vectors can be employed. Adenovirus serotypes 1 through 47 are currently available from American Type Culture Collection (ATCC, Manassas, Va.). However, the application includes any other serotype of adenovirus available from any source. The adenoviruses that can be employed may be of human or non-human origin, such as bovine, porcine, canine, simian, avian. For instance, an adenovirus can be of subgroup A (e.g., serotypes 12, 18, 31), subgroup B (e.g., serotypes 3, 7, 11, 14, 16, 21, 34, 35, 50), subgroup C (e.g., serotypes 1, 2, 5, 6), subgroup D (e.g., serotypes 8, 9, 10, 13, 15, 17, 19, 20, 22-30, 32, 33, 36-39, 42-47, 49, 51), subgroup E (serotype 4), subgroup F (serotype 40, 41), or any other adenoviral serotype, numerous examples of which may be found in the American Type Culture Collection Catalog, found for example at <http://www.atcc.org/SearchCatalogs/CellBiology.cfm>.

The adenovirus or adenoviral vector can refer to the virus itself or derivatives thereof both naturally occurring and recombinant forms, except where indicated otherwise. Such adenoviruses may be wildtype or may be modified in various ways known in the art or as disclosed herein. Such modifications include modifications to the adenovirus genome that is packaged in the particle in order to make an infectious virus. Such modifications include deletions known in the art, such as deletions in one or more of the E1a, E1b, E2a, E2b, E3, or E4 coding regions. The terms adenoviruses and adenoviral vectors also include conditionally replicating adenoviruses (CRADs); that is, viruses that preferentially replicate in certain types of cells or tissues but to a lesser degree or not at all in other types. Preferably, the adenoviral vectors selectively replicate in tumor cells and or abnormally proliferating tissue, such as solid tumors and other neoplasms. These include the viruses disclosed in U.S. Pat. Nos. 5,677,178, 5,698,443, 5,871,726, 5,801,029, 5,998,205, and 6,432,700, the disclosures of which are incorporated herein by reference in their entirety. Such viruses are sometimes referred to as cytolytic or cytopathic viruses (or vectors), and, if they have such an effect on neoplastic cells, are referred to as oncolytic viruses (or vectors). CRADs often contain an essential viral gene such as E1a under the control of or operably linked to, for example, E2F responsive promoters or TERT promoters. A gene essential for replication refers to a nucleic acid sequence whose transcription is required for a viral vector to replicate in a target cell. In the majority of tumor types, the Rb cell cycle regulatory pathway is disrupted. These mutations can be in Rb itself or in other factors that have an effect on upstream regulators of pRB. One consequence of these mutations is the disruption of E2F-pRB binding and an increase in free E2F in tumor cells. The abundance of free E2F in turn results in high level expression of E2F responsive genes in tumor cells. Thus E2F responsive promoters preferentially kill Rb-pathway defective tumor cells as compared to cells which are non-defective in the Rb-pathway. Vectors with a TERT promoter operably linked to a gene essential to replication, preferentially kill tumor cells with up-regulated expression of telomerase as compared to non-tumor cells. As used herein, tumor cell specific regulatory element includes regulatory elements such as promoters that allow the virus to selectively replicate in tumor cells and/or abnormally proliferating tissue. Regulatory elements, typically, are sequences involved in controlling the expression of a nucleotide sequence. Regulatory elements include promoters, enhancers, and termination signals. Regulatory elements also include sequences required for proper translation of the nucleotide sequence.

Herpes Simplex Virus Type-1 and Type-2 Vectors

Herpes simplex virus (HSV) means both type I HSV and type 2 HSV. HSV is a suitable vaccine vector as a substantial number of viral genes are considered non-essential for virus growth in cell culture and consequently large or multiple transgenes can be inserted into the HSV genome without affecting its ability to replicate. Various aspects of the biology of HSV make it attractive as a vaccine vector: broad host cell range, high infectivity, non-dividing cells may be efficiently transduced and made to express transgenes, large capacity for exogenous transgene insertion (approximately half of the viral genes are nonessential for growth in tissue culture and can be replaced by therapeutic transgene cassettes), recombinant replication-defective HSV may readily be prepared to high titer and purity without contamination from wild-type recombinants, and the latent behavior of the virus may be exploited for the stable long-term expression of therapeutic transgenes in neurons.

Generation of replication-incompetent vectors can be accomplished by disruption of one or more essential immediate early (1E) genes. Suitable modifications include, but are not limited to, disruptions of γ₁34.5, thymidine kinase, ribonucleotide reductase, ICP4, ICP0, ICP22, ICP27, and ICP0. Multiple combinations of genes can be disrupted, for example, ICP4, ICP22 and ICP27 can be disrupted. Alternatively, ICP-4, ICP-0, and yl 34.5 can be disrupted. Generally, the disabled vector is manufactured by propagating the virus in a complementing cell line producing the gH glycoprotein. Recombinants are created by recombination of the HSV genome and a transfected plasmid carrying the gene of interest flanked by HSV sequences. A variety of oncolytic HSV vectors have been developed, with three among these, G207, 1716, and NV1020, safely completing phase I clinical trials. G207 is a multigene mutant of HSV-1 that contains deletions of both copies of the 34.5 gene, the major viral determinant of neurovirulence and antagonist of activated double-stranded RNA-dependent protein kinase R, and an Escherichia coli LacZ insertion that inactivates the ICP6 gene (UL39), encoding the large subunit of ribonucleotide reductase, a key enzyme in nucleotide metabolism and viral DNA synthesis in nondividing cells. G47, a derivative of G207, contains an additional deletion of the nonessential α47 gene (ICP47). Because of the overlapping transcripts encoding ICP47 and US11, this deletion places the late US11 gene under control of the immediate-early α47 promoter, which results in an enhancement of growth of γ₁34.5-mutants and broadens the range of susceptible tumor cells by precluding the shutoff of host protein synthesis.

NV1020 (previously called R7020) is a HSV-1/HSV-2 intertypic recombinant. It contains a large deletion of the joint region, including UL56 and one copy of γ₁34.5, a duplication of UL5 and UL6, insertion of HSV-2 gJ, gG, and PK, and deletion of UL24. It has been tested against non-CNS tumors, including colon cancer metastatic to the liver in mice and humans. NV1023 is derived from NV1020 by repairing the thymidine kinase/UL24 region, and insertion of E. coli LacZ into the ICP47 region, deleting ICP47 and US11. This vector was used as the backbone to generate NV1042, which expresses murine interleukin (IL)-12. IL-12 expression has previously been shown to enhance the efficacy of oncolytic HSV therapy and augment the antitumor immune response generated.

Poxvirus Vectors

The poxviridae comprise a large family of complex DNA viruses that replicate in the cytoplasm of vertebrate and invertebrate cells. As used herein, a poxvirus includes any member of the family Poxviridae, including the subfamilies Chordopoxviridae (vertebrate poxviruses) and Entomopoxviridae (insect poxviruses). See, for example, B. Moss in Virology, ed. Fields et al., Raven Press p. 2080 (1990). The chordopoxviruses comprise the following genera: Orthopoxvirus (e.g., vaccinia); Avipoxvirus (e.g., fowlpox); Capripoxvirus (e.g., sheeppox) Leporipoxvirus (e.g., rabbit (Shope) fibroma, myxoma); and Suipoxvirus (e.g., swinepox).

Suitable recombinant vaccinia virus vectors include vectors with a deletion of the DNA encoding a C-terminal portion of the E3L gene product. These viruses maintain viral replication, protein synthesis and interferon-resistance that is indistinguishable from wild-type virus, but these viruses have remarkably reduced pathogenicity in mice relative to wild-type vaccinia virus of the same strain.

Vaccinia virus has demonstrated physical and genetic stability under field conditions, reducing problems and expense in transport and storage. Recombinant vaccinia virus vectors have been shown to confer cellular and humoral immunity against foreign gene products and to protect against infectious diseases in several animal models. Further, recombinant vaccinia viruses have also been used in clinical trials to express the gp160 envelope gene of HIV and are thus clinically accepted.

Suitable vaccinia viruses also include, but are not limited to, the Copenhagen (VC-2) strain, modified Copenhagen strain (NYVAC), the WYETH strain and the modified Ankara (MVA) strain. Other poxviruses suitable for use as vectors may be used. For example, fowlpox strains such as ALVAC and TROVAC vectors also provide desirable properties and are highly attenuated. Methods and conditions for constructing recombinant poxvirus virus vectors, such as vaccinia virus vectors, are known in the art.

Several properties of vaccinia virus render it well suited to use as a viral vector. First, vaccinia virus exhibits tropism for a wide range of mammalian cell types. In addition, its nearly 200-kilobase (kb) genome allows for the delivery of large transgene sequences. Although vaccinia virus administration elicits a vigorous immune response, this immunogenicity can be exploited to augment host immunity against tumor cells. Vaccinia virus mutants that replicate conditionally have been developed, such that they destroy human cancer cells as a byproduct of viral replication. The strategy most commonly used involves deletion or insertional inactivation of the vaccinia virus TK gene, which inhibits viral replication in normal, nondividing cells but allows viral replication in cells with large intracellular nucleotide pools, such as tumor cells. Another strategy involves deletion of the SPI-1 and SPI-2 genes from the vaccinia viral genome. These genes encode viral serine proteases and are required for robust viral replication. However, tumor cells overexpress homologous proteins, and, therefore, vaccinia virus mutants defective in SPI-1 and SPI-2 replicate preferentially in these tumor cells rather than normal cells.

Lentiviral Vectors

Lentiviruses belong to the retrovirus family, but they can infect both dividing and non-dividing cells. The lentivirus group can be split into primate and non-primate. Examples of primate lentiviruses include the human immunodeficiency virus (HIV) and the simian immunodeficiency virus (SIV). The non-primate lentiviral group includes visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

Details on the genomic structure of some lentiviruses may be found in the art. By way of example, details on HIV and E1AV may be found from the NCBI Genbank database (i.e. Genome Accession Nos. AF033819 and AF033820 respectively). Examples of HIV-1 variants may be found in the HIV databases maintained by Los Alamos National Laboratory. Details of ELAV clones may be found at the NCBI database maintained by the National Institutes of Health.

Recombinant lentiviral vectors refer to vectors with sufficient genetic information to allow packaging of an RNA genome, in the presence of packaging components, into a viral particle capable of infecting and transducing a target cell. Infection and transduction of a target cell includes reverse transcription and integration into the target cell genome. The vector carries non-viral coding sequences which are to be delivered by the vector to the target cell. A recombinant lentiviral vector is incapable of independent replication to produce infectious retroviral particles within the final target cell. Usually the vector lacks a functional gag-pol and/or env gene and/or other genes essential for replication. Preferably the recombinant lentiviral vector has a minimal viral genome. The term minimal viral genome means that the viral vector has been manipulated so as to remove the non-essential elements and to retain the essential elements in order to provide the required functionality to infect, transduce and deliver a nucleotide sequence of interest to a target host cell.

Alternative sequences which perform the same function as the rev/RRE system are also known. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. It is also known that the Rex protein of HTLV-1 can functionally replace the Rev protein of HIV-1. It is also known that Rev and Rex have similar effects to IRE-BP.

The lentiviral vector is derived from a non-primate lentivirus. The non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV). E1AV has the simplest genomic structure of the lentiviruses. In addition to the gag, pol and env genes E1AV encodes three other genes: tat, rev, and S2. Tat acts as a transcriptional activator of the viral LTR and Rev regulates and coordinates the expression of viral genes through rev-response elements (RRE). The mechanisms of action of these two proteins are thought to be broadly similar to the analogous mechanisms in the primate viruses.

Lentiviral vectors include primate lentiviral vectors such as HIV vectors (for example, HIV-1 and HIV-2 vectors) and SIV vectors, and non-primate lentiviral vectors. Primate lentiviral vectors have a number of disadvantages which may limit their therapeutic application to certain diseases. For example, HIV-1 has the disadvantage of being a human pathogen carrying potentially oncogenic proteins and sequences. There is the risk that introduction of vector particles produced in packaging cells which express HIV gag-pol will introduce these proteins into an individual, leading to seroconversion. Therefore, in a particularly preferred embodiment, the lentiviral vector will be a non-primate lentiviral vector, such as E1AV, FIV, BIV, CAEV or MVV, with E1AV being especially preferred. Non-primate lentiviral-based vectors do not introduce HIV proteins into primates.

Lentiviral vectors, such as those based on primate lentiviruses, (e.g., HIV and SIV) offer several advantages over retroviral vectors. Lentiviral vectors containing genes encoding antibodies can infect quiescent cells and proliferating cells both in vivo and in vitro. Additionally, lentiviral vectors allow constitutive or induced expression of heterologous polypeptides, thus providing for the production of antibodies in culture and in animal.

Although the HIV-1 vector systems have been the most extensively studied, other lentivirus vectors also have been developed. HIV-2 is genetically more closely related to SIV than to HIV-1, is less pathogenic in humans, and can be studied in a primate animal model. Furthermore, in certain situations, HIV-2 vectors may be preferable to HIV-1 vectors for delivering therapeutic genes. In addition to HIV-2 and SIV vectors, chimeric vector systems composed of HIV-1 and HIV-2 components or of HIV-1 and SIV components have been described.

Spumavirus Vectors

Spumavirus vectors or foamy viral (FV) vectors have a nonpathogenic nature, even in cases of accidental human infection, which is a major feature that makes FVs attractive vector systems. Such systems consist of two components, (i) the vector proper, harboring in cis the genetic elements required for RNA (pre-)genome dimerization and packaging, reverse transcription, provirus integration, and transgene expression, and (ii) a packaging cell line supplying the viral Gag, Pol, and Env proteins in trans.

Cytomegalovirus Vectors

Cytomegalovirus (CMV) is a genus of Herpes viruses; in humans the species is known as Human herpesvirus 5 (HHV-5). It belongs to the Betaherpesvirinae subfamily of Herpesviridae. CMV include, but are not limited to, Cercopithecine herpesvirus 5 (CeHV-5)—African green monkey cytomegalovirus, Cercopithecine herpesvirus 8 (CeHV-8)—Rhesus monkey cytomegalovirus, Human herpesvirus 5 (HHV-5)—Human cytomegalovirus, Pongine herpesvirus 4 (PoHV-4). Aotine herpesvirus 1 (AoHV-1)—Herpesvirus aotus 1 and Aotine herpesvirus 3 (AoHV-3)-Herpesvirus aotus 3 are also thought to belong to the family of CMV.

Cytomegalovirus (CMV) is a suitable viral vector because of the ability of its genome to persist in hematopoietic progenitor cells and the packaging capacity of the viral capsid that accommodates a DNA genome of 230 kbp. Amplicons are herpesviral vectors, which contain only the cis-active sequences required for replication and packaging of the vector genome. For construction of a CMV amplicon the sequences comprising the lytic origin of replication (orilyt) and the cleavage packaging recognition sites (pac) of human CMV are usually cloned onto a plasmid. The amplicon plasmid replicates in the presence of a CMV helper virus and is packaged into CMV particles, with replication and packaging being dependent on the presence of the orilyt and pac sequences. The packaged amplicon can be transferred to recipient cells and reisolated from the transduced cells. The CMV amplicon vector has the potential to transfer therapeutic genes with a size of more than 200 kbp.

Each of the vectors described above can comprise a mutant DNA polymerase active at high dNTP concentrations. As described above, the viral vector can also comprise an essential viral gene operably linked to a tumor specific promoter. Optionally, the viral vector can further comprise alone or in combination with other genes, a gene that functionally complements an essential viral gene, wherein the essential viral gene is non-functional. As used herein an essential viral gene means a gene that is essential for viral replication. Thus, an essential viral gene refers to a nucleic acid sequence whose transcription is required for a viral vector to replicate in a target cell.

All of the vectors described above can also comprise additional nucleic acids. Thus it will be appreciated that the vectors may include any of a number of genes of interest, combinations of genes of interest and genes of interest/regulatory element combinations. Preferably the gene is a therapeutic gene. The term therapeutic gene is used herein to refer to a foreign or endogenous gene that expresses a molecule that has a therapeutic effect on the host. The therapeutic genes can be used singly or in any desired combination.

Suitable therapeutic genes include, but are not limited to, genes that may sensitize tumor tissues to inducible forms of cell death, or increase the efficiency of adenovirally-mediated cell killing including thymidine kinase, the sodium-iodide symporter [NIS], products of the adenovirus E3 gene and other genes. Suitable therapeutic genes also include genes that may enhance immunologic destruction of tumor tissues including tumor-specific antigens such as alpha-fetoprotein, her2, CYP1A1, cytokines or interferons such as GMCSF, TNF-alpha, IFN-gamma, chemokines such as MIP3-alpha, costimulatory molecules such as CD40, CD40L, CD80, CD86, or allogeneic HLA molecules such as HLA B7.

A nucleic acid molecule encoding a prodrug may be used. Most typically, a prodrug activates a compound with little or no cytotoxicity into a toxic compound. Prodrugs include, but are not limited to, nitroreductase, thymidine kinase and cytosine deaminase. Gene products that may be utilized include E. coli guanine phosphoribosyl transferase, which converts thioxanthine into toxic thioxanthine monophosphate; alkaline phosphatase, which converts inactive phosphorylated compounds such as mitomycin phosphate and doxorubicin-phosphate to toxic dephosphorylated compounds; fungal (e.g., Fusarium oxysporum) or bacterial cytosine deaminase, which converts 5-fluorocytosine to the toxic compound 5-fluorouracil; carboxypeptidase G2, which cleaves glutamic acid from para-N-bis(2-chloroethyl) aminobenzoyl glutamic acid, thereby creating a toxic benzoic acid mustard; and Penicillin-V amidase, which converts phenoxyacetabide derivatives of doxorubicin and melphalan to toxic compounds. Moreover, a wide variety of Herpesviridae thymidine kinases, including both primate and non-primate herpesviruses, are suitable. Other gene products may render a cell susceptible to toxic agents. Such products include tumor necrosis factor, viral proteins, and channel proteins that transport drugs. A cytocide-encoding agent may be constructed as a prodrug, which, when expressed in the proper cell type, is processed or modified to an active form. For example, the saporin gene may be constructed with an N- or C-terminal extension containing a protease-sensitive site. The extension renders the protein inactive and subsequent cleavage in a cell expressing the appropriate protease restores enzymatic activity.

The viral vector optionally encodes tumor suppressor genes. Examples of the foregoing include cell adhesion molecules such as E-cadherin, which play a role in tissue development and epithelial cell differentiation and human BGP (biliary glycoprotein). Other tumor-suppressor genes useful include the following Rb, p53, CDKN2/P16/MTS1, PTEN/MMAC1, APC, 331NG1, Smad4, maspin, von Hippel-Lindau (VHL), Wilms tumor (WTI), Bin1, Men1, Neurofibromatosis 2 (NF2), MXI1 and FHIT. It should be noted, however, that this list is not exhaustive, only exemplary.

The viral vectors optionally also encode therapeutic gene products involved in vascularization, wound healing and tissue repair.

The viral vectors optionally also comprise nucleic acids that encode apoptosis inducing agents. Examples of nucleotide sequences which encode such agents include, but are not limited to, p53, c-myc, TNF-alpha, Fas ligand, TRAIL, p38-mitogen activated protein (MAP) kinase and IFN-gamma.

The viral vectors optionally comprise cytocidal gene products. A cytocide-encoding agent is a nucleic acid molecule (e.g., DNA or RNA) that, upon internalization by a cell, and subsequent transcription (if DNA) and[/or] translation into a cytocidal agent, is cytotoxic or cytostatic, to a cell, for example, by inhibiting cell growth through interference with protein synthesis or through disruption of the cell cycle. Cytocides include saporin, the ricins, abrin, gelonin, other ribosome inactivating proteins, Pseudomonas exotoxin, diphtheria toxin, angiogenin, tritin, dianthins 32 and 30, momordin, pokeweed antiviral protein, mirabilis antiviral protein, bryodin, angiogenin, and shiga exotoxin, as well as other cytocides that are known to those of skill in the art.

The viral vectors optionally comprise therapeutic genes encoding cell cycle inhibitors including DNA molecules that encode an enzyme that results in cell death or renders a cell susceptible to cell death upon the addition of another product. For example, saporin is an enzyme that cleaves rRNA and inhibits protein synthesis. Other enzymes that inhibit protein synthesis are especially well suited for use in the present invention. Alternatively, the product may be a ribozyme, antisense, or other nucleic acid molecule that causes cell death.

The viral vectors optionally comprise promoters and additional elements as desired. A therapeutic gene is generally in operative linkage with an appropriate promoter for expression of polypeptides in recipient cells. Thus, the therapeutic genes, promoters and additional elements can be used in any combination in any of the viral vectors described herein. In general, viral vectors will also contain elements necessary for transcription and translation. The choice of the promoter will depend upon the cell type to be transformed and the degree or type of control desired. Promoters can be constitutive or active in any cell type, tissue specific, cell specific, event specific, temporal-specific or inducible. Cell-type specific promoters and event type specific promoters are preferred. Examples of constitutive or nonspecific promoters include the SV40 early promoter, the SV40 late promoter, CMV early gene promoter and adenovirus promoter. Tissue specific promoters are particularly useful for expression in a wide variety of cells. SMC-specific promoters are particularly useful in targeting proliferative diseases involving SMC. For example, FGFR promoter, EGFR promoter, PDGF receptor promoter, integrin receptor promoters, α-actin promoter, SM1 and SM2 myosin heavy chain promoters, calponin-h1 promoter and SM22 alpha angiotensin receptor promoter.

Exemplary tissue-specific promoters also include alpha-crystalline, tyrosinase, α-fetoprotein, prostate specific antigen, CEA, α-actin, VEGF receptor, erbB-2, C-myc, cyclin D, FGF receptor, gamma-crystalline promoter, tek, tie, urokinase receptor, E-selectin, P-selectin, VCAM-1, endoglin, endosialin, αᵥ integrin, β₃ integrin, endothelin-1, ICAM-3, E9, von Willebrand Factor, CD-44, CD40, vascular endothelial cadherin, notch 4 and high molecular weight melanoma-associated antigen.

Inducible promoters are optionally used. These promoters include MMTV LTR, inducible by dexamethasone, metallothionein, inducible by heavy metals, and promoters with cAMP response elements, inducible by cAMP, as well as radiation-inducible promoters, inducible by therapeutic irradiation. By using an inducible promoter, the nucleic acid may be delivered to a cell and will remain quiescent until the addition of the inducer. This allows further control on the timing of production of the gene product.

Event-type specific promoters are active or up-regulated only upon the occurrence of an event, such as tumorigenicity or viral infection. Additionally, promoters that are coordinately regulated with a particular cellular gene may be used.

Elements that increase the expression can be incorporated into the viral vector. Such elements include internal ribosome binding sites (IRES) as well as other sequences that may enhance expression, including self-cleaving proteolytic elements or nonsense codons that can be selectively read-through following administration of an exogenous substance such as gentamycin. Other elements may be incorporated into the vector including a polyadenylation sequence, splice donor and acceptor sites, and an enhancer. These elements can be used in combination with promoters and/or therapeutic genes.

Methods of Making

The compositions disclosed herein and the compositions necessary to perform the disclosed methods are made using any method known to those of skill in the art for that particular reagent or compound unless otherwise specifically noted. For example, nucleic acids are optionally made using standard chemical synthesis methods or can be produced using enzymatic methods or any other known method. Such methods can range from standard enzymatic digestion followed by nucleotide fragment isolation (see, for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System IPlus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

The viral vectors can be made recombinantly as set forth in the examples or by other methods of making recombinant viruses as described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Similar methods are used to introduce a gene of interest in methods of making the viral vector described herein. For example, recombinant viruses are optionally constructed using homologous recombination after DNA co-transfection. In this example, cells are co-transfected with at least two different viruses containing the genes of interest and progeny virus plaque can be purified based upon loss of marker expression. Final verification of the correct genetic organization of candidate viruses are verified by DNA hybridization studies using probes to the nucleic acids as described herein.

Nucleic acid sequences described herein are obtained using standard cloning and screening techniques, from natural sources such as genomic DNA libraries or can be synthesized using well known and commercially available techniques.

When nucleic acid sequences are used recombinantly, the nucleic acid sequence optionally include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. Nucleic acid sequence may also contain non-coding 5′ and 3′ sequences, such as transcribed, non-translated sequences, splicing and polyadenylation signals, ribosome binding sites and sequences that stabilize mRNA.

Compositions

Provided are also pharmaceutical compositions comprising a viral vector comprising a nucleic acid encoding a mutant viral DNA polymerase. Thus the herein provided viruses and viral vectors can be administered in vitro or in vivo in a pharmaceutically acceptable carrier. By pharmaceutically acceptable is meant a material that is not biologically or otherwise undesirable, i.e., the material may be administered to a subject, along with the vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials are optionally in solution, suspension (for example, incorporated into microparticles, liposomes, or cells). These are optionally targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as stealth and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. For review, see Brown and Greene, DNA and Cell Biology 10:6, 399-409 (1991).

Pharmaceutical compositions optionally include carriers, thickeners, diluents, buffers, preservatives, surface active agents and the like in addition to the molecule, in this case virus or viral vector, of choice. Pharmaceutical carriers are known to those skilled in the art. These most typically are standard carriers for administration of drugs to humans, including solutions such as sterile water, saline, and buffered solutions at physiological pH. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of a pharmaceutically-acceptable carriers include, but are not limited to, saline, Ringer's solution and dextrose solution. Further carriers may include sustained release preparations such as semipermeable matrices of solid hydrophobic polymers containing the antibody, which matrices are in the form of shaped articles, e.g., films, liposomes or microparticles. Certain carriers may be more preferable depending upon, for instance, the route of administration and concentration of composition being administered.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Some of the compositions are administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines. The various pharmaceutically acceptable compositions and components thereof are used with any of the viral vectors described herein.

Methods of Administration

Generally viral particles are transferred to a biologically compatible solution or pharmaceutically acceptable delivery vehicle, such as sterile saline, or other aqueous or non-aqueous isotonic sterile injection solutions or suspensions, numerous examples of which are well known in the art, including Ringer's, phosphate buffered saline, or other similar vehicles. Delivery of the viral vector is carried out via any of several routes of administration including topical, oral, by inhalation, or parenterally, for example by intravenous drip, subcutaneous, intraperitoneal or intramuscular injection. Thus, the disclosed viruses and vectors are administered, for example, intravenously, intraperitoneally, intramuscularly, subcutaneously, intracavity, transdermally, intrahepatic administration, catheterization (including cardiac catheterization), intracranial injection, nebulization/inhalation or by instillation via bronchoscopy.

The viral vectors are administered in a number of ways depending on whether local or systemic treatment is desired, and on the area to be treated. For example, the provided viral vectors are administered directly into a tumor by stereotactic delivery. It is also understood that delivery to tumors of the CNS is, for example, by intravascular delivery if the virus or vector is combined with a moiety that allows for crossing of the blood brain barrier and survival in the blood. Thus, agents are combined to increase the permeability of the blood brain barrier. Agents include, for example, elastase and lipopolysaccharides. The provided viruses and vectors are administered via the carotid artery. In another aspect, the provided viruses and vectors are administered in liposomes, such as those known in the art or described herein. The provided viruses and vectors are administered to cancers not in the brain intravascularly, intraperitoneally or by direct injection into the tumor, for example.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. Sec, e.g., U.S. Pat. No. 3,610,795, which is incorporated by reference herein in its entirety for the methods taught therein.

It is also possible to link molecules (e.g., to form viral vector conjugates) to viruses or viral vectors to enhance, for example, cellular uptake. Conjugates are chemically linked to the virus or viral vector. Such conjugates include but are not limited to lipid moieties such as a cholesterol moiety. (Letsinger et al., Proc. Natl. Acad. Sci. USA, 1989, 86:6553-6556).

The viruses and viral vectors described herein are administered, for example, by convection enhanced delivery, which has been used with adenovirus to increase the distribution of the virus thorough bulk flow in the tumor interstitium. Genetic modifications have also been used to enhance viral spread. For example, insertion of the fusogenic glycoprotein gene produced an oncolytic virus with enhanced antiglioma effect. Therefore, the viral vectors described herein may comprise such a gene.

Dosages

Optimal dosages of DNA or virus depend on a variety of factors and may thus vary somewhat from subject to subject. Therapeutically effective doses of viruses are considered to be in the range of about 1×10⁴ to about 1×10¹⁰ pfu of virus/ml, including 10⁴, 10⁵, 10⁶, 10⁷, 10⁸, 10⁹ and 10¹⁰ pfu, or any amount in between.

The exact amount required varies from subject to subject, depending on the species, age, weight and general condition of the subject, the severity of the disease being treated, the particular virus or vector used and its mode of administration. Thus, it is not possible to specify an exact amount for every composition. However, an appropriate amount is determined by one of ordinary skill in the art using only routine experimentation given the guidance provided herein.

Effective dosages and schedules for administering the compositions are determined empirically, and making such determinations is within the skill in the art. For example, animal models for a variety of cancers are obtained, for example, from Jackson Laboratory, 600 Main Street, Bar Harbor, Me. 04609 USA, which provides hundreds of cancer mouse models. Both direct (e.g., histology of tumors) and functional measurements (e.g., survival of a subject or size of a tumor) can be used to monitor response to oncolytic therapy. These methods involve the sacrifice of representative animals to evaluate the population, increasing the animal numbers necessary for the experiments. Measurement of luciferase activity in the tumor provides an alternative method to evaluate tumor volume without animal sacrifice and allowing longitudinal population-based analysis of therapy.

The dosage ranges for the administration of the compositions are those large enough to produce the desired effect in which the symptoms of the disease are affected. The dosage should not be so large as to cause adverse side effects, such as unwanted cross-reactions and anaphylactic reactions. The dosage is adjusted by the individual physician in the event of any counterindications. Dosage varies and is administered, for example, in one or more dose administrations daily, for one or several days.

Viral recovery and immunohistochemistry have been used successfully to monitor viral replication and spread in vivo. Bioluminescent and fluorescent protein expression by the virus, for example, is used to indirectly monitor viral replication and spread in the tumor. Genes encoding fluorescent reporter proteins (d2EGFP and dsRED monomer) or bioluminescent markers (firefly luciferase) are commonly used in recombinant viruses. Not only do these facilitate the screening and selection of recombinant viruses in vitro. The reporter genes also allow indirect monitoring of viral activity in the in vivo studies. The various methods of administration can be used with any of the viral vectors and pharmaceutically acceptable compositions and components thereof. One of skill in the art selects the vectors, pharmaceutical compositions and components according to the route of administration.

Diseases to be Treated

Methods of treating cancer in a subject comprising administering a viral vector comprising a mutant DNA polymerase are described. As used herein the terms treatment, treat or treating refers to a method of reducing the effects of a disease or condition or symptom of the disease or condition. Thus in the disclosed method treatment can refer to a 10%, 20%, 30%, 40%, 50%, 60%, 70%, 80%, 90% or 100% reduction in the severity or rate of progression of an established disease or condition or symptom of the disease or condition or a delay in onset of one or more symptoms. For example, the method for treating cancer is considered to be a treatment if there is a 10% reduction in one or more symptoms or in the delay of one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, subject includes a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be covered. As used herein, patient or subject may be used interchangeably and can refer to a subject afflicted with a disease or disorder. The term patient or subject includes human and veterinary subjects.

Cancer, cancer cells, neoplastic cells, neoplasia, tumor, and tumor cells are used interchangeably to refer to cells that exhibit relatively autonomous growth, so that they exhibit an aberrant growth phenotype characterized by a significant loss of control of cell proliferation. Neoplastic cells include malignant or benign cells.

Neoplasms include solid tumors, for example, carcinomas and sarcomas. Carcinomas include malignant neoplasms derived from epithelial cells which infiltrate or invade surrounding tissues and give rise to metastases. Adenocarcinomas are carcinomas derived from glandular tissue, or from tissues that form recognizable glandular structures. Another broad category of cancers includes sarcomas and fibrosarcomas, which are tumors whose cells are embedded in a fibrillar or homogeneous substance, such as embryonic connective tissue. Provided herein are methods of treatment of cancers of myeloid or lymphoid systems, including leukemias, lymphomas, and other cancers that typically are not present as a tumor mass but are distributed in the vascular or lymphoreticular systems.

Also provided are methods for treatment of adult and pediatric cancer; growth of solid tumors/malignancies; myxoid and round cell carcinoma; locally advanced tumors; human soft tissue sarcomas, including Ewing's sarcoma; cancer metastases, including lymphatic metastases; squamous cell carcinoma, particularly of the head and neck, esophageal squamous cell carcinoma; oral carcinoma; blood cell malignancies including multiple myeloma; leukemias, including acute lymphocytic leukemia, acute nonlymphocytic leukemia, chronic lymphocytic leukemia, chronic myelocytic leukemia, and hairy cell leukemia; effusion lymphomas, including body cavity based lymphomas; thymic lymphoma lung cancer, including small cell carcinoma of the lungs; cutaneous T cell lymphoma; Hodgkin's lymphoma; non-Hodgkin's lymphoma; cancer of the adrenal cortex; ACTH-producing tumors; non-small cell lung cancers; breast cancer, including small cell carcinoma and ductal carcinoma; gastro-intestinal cancers, including stomach cancer, colon cancer, colorectal cancer, and polyps associated with colorectal neoplasia; pancreatic cancer; liver cancer; urological cancers, including bladder cancer, such as primary superficial bladder tumors, invasive transitional cell carcinoma of the bladder, and muscle-invasive bladder cancer; prostate cancer; malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle; malignancies of the male genital tract, including testicular cancer and penile cancer; kidney cancer, including renal cell carcinoma; brain cancer, including intrinsic brain tumors, neuroblastoma, astrocytic brain tumors, gliomas, and metastatic tumor cell invasion in the central nervous system; bone cancers, including osteomas and osteosarcomas; skin cancers, including malignant melanoma, tumor progression of human skin keratinocytes, basal cell carcinoma, and squamous cell cancer; thyroid cancer; retinoblastoma; neuroblastoma; peritoneal effusion; malignant pleural effusion; mesothelioma; Wilms's tumors; gall bladder cancer; trophoblastic neo-plasms; hemangiopericytoma; and Kaposi's sarcoma.

Combination Therapy

Valproic acid (VPA) effects on adenovirally-mediated gene expression and virus replication. Valproic acid (VPA) has a long history as a safe and well-tolerated drug used in the treatment of mood and seizure disorders. VPA is also an inhibitor of class I cellular histone deacetylases (HDACs) and can trigger the proteasomal degradation of class II HDACs. HDAC1 is known to regulate HIV-1 latency within resting CD4+ T cells, which can persist for years or even decades. VPA helps drive virus reactivation from latently infected, resting CD4+ T cells, with the aim of accelerating the clearance of this virus reservoir in patients who were receiving an intensified anti-retroviral regimen (Lehrman, et al., Lancet 366:549-55 (2005)). VPA (because of its favorable safety and tolerability profile) helps inhibit HDAC activity in cancer cells, and thereby induces tumor cell differentiation or growth arrest (Chavez-Blanco, et al., Mol. Cancer. 4:22 (2005)).

VPA also mediates effects on virally-vectored gene transfer and virus replication. Thus, VPA treatment results in enhanced replication and/or reactivation from latency of both DNA (cytomegalovirus, human herpesvirus-8/KSHV) and RNA (HIV-1) viruses. VPA also increases gene expression from replication defective Ad5 vectors both in vitro and in vivo (Fan, et al., J. Virol. Methods 125:23-33 (2005)). In fact, HDAC inhibitors such as VPA are useful in the context of oncolytic CRADs based on adenovirus type 5 (Ad5). This is because other HDAC inhibitors (which lack VPA's long track record of safe human use) have been shown to lead to increased expression of both the primary receptor for Ad5 (the coxsackie-and-adenovirus receptor; CAR) and of the viral integrin coreceptors (αv integrin) on cancer cell lines (6/6 cell lines tested, from a broad range of different cancer types). Therefore, VPA may exert a synergistic effect on oncolytic CRADs by increasing viral replication and enhancing virally directed gene expression.

For example, VPA or other HDAC inhibitors (including, for example, FK228 (depsipeptide, formally named FR901228); vorinostat (suberoylanilide hydroxamic acid or SAHA), LAQ-824, MS-275, LBH589, or BL1521) are optionally used in combination with any of the viral vectors described herein. Furthermore, such inhibitors are optionally combined with any of the pharmaceutical compositions or components thereof.

Combination regimens of treatment for use in the methods described herein include, but are not limited to, the CRADs described herein and/or VPA in combination with chemotherapy, radiotherapy and immune therapy. Immune therapy as used herein includes therapeutic vaccination, cytokine delivery, depletion of regulatory T cells and other gene transfer/gene therapy modalities.

Thus, the viral vectors comprising a mutant DNA polymerase described herein are optionally administered alone or in combination with one or more therapeutic agents of therapeutic regimen(s). Such combinations include any other agent(s) described herein that can be used with the viral vector. The therapeutic agents include but are not limited to other members of the TNF family, chemotherapeutic agents, antibodies, antivirals, steroidal and non-steroidal anti-inflammatories, conventional immunotherapeutic agents, cytokines, chemokines, and/or growth factors. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents.

Members of the TNF family include but are not limited to soluble forms of TNF-α, lymphotoxin-α (LT-α, also known as TNF-β), LT-β (found in complex heterotrimer LT-α2-β) OPGL, FasL, CD27L, CD30L, CD40L, 4-1BBL, DcR3, OX40L, TNF-γ, TRAIL, AIM-II, APRIL, endokine-α, TR6, OPG and nerve growth factor (NGF), and soluble forms of Fas, CD30, CD27, CD40 and 4-IBB, TR2, DR3, DR4, TR5, TRANK, TR9, TR10, 312C2, TR12, and soluble forms of CD154, CD70, and CD153.

The viral vector are optionally administered in combination with one or more of tacrolimus, thalidomide, anti Tac (Fv)-PE40, inolimomab, MAK-195F, ASM-981, interleukin-1 receptor, interleukin-4 receptor, ICM3, BMS-188667, anti-TNF Ab, CG-1088, anti-B7 monoclonal antibody, MEDI-507 and ABX-CBL.

The viral vectors are optionally administered in combination with anti-retroviral agents, nucleoside reverse transcriptase inhibitors, non-nucleoside reverse transcriptase inhibitors, and/or protease inhibitors. Nucleoside reverse transcriptase inhibitors include, but are not limited to, RETROVIR® (zidovudine/AZT) (GaxoSmithKline, Research Triangle Park, N.C.), VIDEX® (didanosine/ddI) (Bristol-Myers Squibb, N.Y.), HIVID® (zalcitabine/ddC) (Roche, Nutley, N.J.), ZERIT® (stavudine) (Bristol-Myers Squibb). Non-nucleoside reverse transcriptase inhibitors that may be administered include but are not limited to VIRAMUNE® (nevirapine) (Boehringer Ingelheim/Roxanne, Columbus, Ohio), RESCRIPTOR® (delavirdine) (Pharmacia & Upjohn Company, Kalamazoo, Mich.), and SUSTIVA® (efavirenz) (Bristol-Myers Squibb).

Protease inhibitors include but are not limited to CRIXIVAN® (indinavir sulfate)(Merck & Company, Whitehouse Station, N.J.), NORVIR® (ritonavir)(Abbott Laboratories, Chicago, Ill.), INVIRASE® (saquinavir)(Roche Pharmaceuticals, Nutley, N.J.), and VIRACEPT® (nelfinavir)(Agouron Pharmaceuticals, San Diego, Calif.).

The viral vector are optionally administered in combination with an antiviral agent. Antiviral agents that may be administered include, but are not limited to, acyclovir, ribavirin, amantadine, and remantidine.

The viral vectors are optionally administered in combination with an antibacterial agent. Antibacterial agents that may be administered include, but are not limited to, amoxicillin, aminoglycosides, beta-lactam (glycopeptide), betalactamases, clindamycin, chloramphenicol, cephalosporins, ciprofloxacin, erythromycin, fluoroquinolones, macrolides, metronidazole, penicillins, quinolones, ritampin, streptomycin, sulfonamide, tetracyclines, trimethoprim, trimethoprim-sulfamthoxazole, and vancomycin.

Conventional nonspecific immunosuppressive agents include, but are not limited to steroids, cyclosporine, cyclosporine analogs, cyclophosphamide methylprednisone, prednisone, azathioprine, FK-506 (Fujisawa Pharmaceuticals, Deerfield, Ill.), 15-deoxyspergualin, and other immunosuppressive agents that act by suppressing the function of responding immune cells (including, for example, T cells), directly (e.g., by acting on the immune cell) or indirectly (by acting on other mediating cells).

The viral vectors are optionally administered in combination with immunosuppressants. Immunosuppressants include, but are not limited to, ORTHOCLONE® (OKT3) (Ortho Biotech, Raritan, N.J.), SANDIMMUNE® ORAL (cyclosporine) (Sandoz Pharmaceuticals, Hanover, N.J.), PROGRAF® (tacrolimus) (Fujisawa Pharmaceuticals, Deerfield, Ill.), CELLCEPT® (mycophenolate) (Roche Pharmaceuticals, Nutley, N.J.), azathioprine, glucorticosteroids, and RAPAMUNE® (sirolimus) (Wyeth, Collegeville, Pa.).

The viral vectors are optionally administered in combination with an anti-inflammatory agent. Anti-inflammatory agents include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, ninesulide, orgotein, oxaceprol, paranyline, perisoxal, pifoxime, proquazone, proxazole, and tenidap.

The viral vectors are optionally administered in combination with an antimalarial including, but not limited to, hydroxychloroquine, chloroquine, and/or quinacrine.

The viral vectors are optionally administered in combination with NSAIDs including, but not limited to, NRD-101 (Hoechst Marion Roussel, Kansas City, Mo.), diclofenac (Dimethaid, Ontario, Conn.), oxaprozin potassium (Monsanto), mecasermin (Chiron, Emeryville, Calif.), T-614 (Toyama), pemetrexed disodium (Eli Lilly, Indianapolis, Ind.), atreleuton (Abbott, Chicago, Ill.), valdecoxib (Monsanto), eltenac (Byk Gulden, Melville, N.Y.), campath, AGM-1470 (Takeda, Lincolnshire, Ill.), CDP-571 (Celltech, Rochester, N.Y.), CM-101 (CarboMed, Nashville, Tenn.), ML-3000 (Merck), CB-2431 (KS Biomedix, Surrey, UK), CBF, BS2 (KS Biomedix, Surrey, UK), IL-Ira gene therapy (Valentis, Burlingame, Calif.), JTE-522 (Japan Tobacco, Tokyo, Japan), paclitaxel (Angiotech, Vancouver, BC), DW-166HC (Dong Wha, Seoul, Korea), darbufelone mesylate (Pfizer Inc., New York, N.Y.), soluble TNF receptor I (synergen; Amgen, Thousand Oaks, Calif.), IPR 6001 (Institute for Pharmaceutical Research), trocade (Hoffman-La Roche, Nutley, N.J.), EF5 (Scotia Pharmaceuticals), BIIL-284 (Boehringer Ingelheim, Ridgefield, Conn.), BIIF-1149 (Boehringer Ingelheim, Ridgefield, Conn.), LEUKOVAX® (Inflammatics, Malvem, Pa.), MK-663 (Merck, Whitehouse Station, N.J.), ST-1482 (Sigma-Tau, Gaithersburg, Md.), and butixocort propionate (Pfizer Inc., New York, N.Y.).

The viral vector is optionally administered in combination with one or more chemotherapeutic agents. Chemotherapeutic agents include, but are not limited to, antibiotic derivatives (e.g. doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2B, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotant, vincristine sulfate, vinblastine sulfate, and etoposide). Others chemotherapeutics include, e.g., CPT-11, deflunomide, cycloheximide, carboplatin, cisplatin, colchicine, diethylstilbestrol, floxuridine, melphalan, 6-mercaptopurine, teniposide, and 6-thioguanine.

The viral vectors are optionally administered in combination with cytokines including, but not limited to, GM-CSF, G-CSF, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, anti-CD40, CD40L, IFN-alpha, IFN-beta, IFN-gamma, TNF-alpha, and TNF-beta.

The viral vectors are optionally administered in combination with one or more other therapeutic or prophylactic regimens, such as, for example, radiation therapy.

Chemotherapeutic agents, antibiotics, anti-inflammatories, anti-virals and immunotherapeutics are optionally administered in combination with the viral vectors described herein. Radiation therapy is optionally administered in combination with the viral vectors. Any of the aforementioned treatments are used in any combination with the viral vectors described herein. Thus, for example, the viral vectors are administered in combination with a chemotherapeutic agent and radiation. Other combinations are administered as desired by those of skill in the art.

It must be noted that, as used in the specification and the appended claims, the singular forms a, an and the include plural referents unless the context clearly dictates otherwise. Thus, for example, reference to an antigenic fragment includes mixtures of antigenic fragments, reference to a pharmaceutical carrier or adjuvant includes mixtures of two or more such carriers or adjuvants.

Optional or optionally means that the subsequently described event or circumstance can or cannot occur, and that the description includes instances where the event or circumstance occurs and instances where it does not. For example, the phrase optionally means a characteristic may or may not be present. Thus, if a composition optionally comprises a combination, the composition may comprise a combination of different molecules or may not include a combination such that the description includes both the combination and the absence of the combination (i.e., individual members of the combination).

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a therapeutic agent or treatment regime is disclosed and discussed and a number of modifications that can be made to the agent or regime are discussed, each and every combination and permutation of the agent and the regime are specifically contemplated unless specifically indicated to the contrary. Likewise, any subset or combination of these is also specifically contemplated and disclosed.

Unless defined otherwise, all technical and scientific terms used herein have the same meanings as commonly understood by one of skill in the art to which the disclosed method and compositions belong. Publications cited herein and the material for which they are cited are hereby specifically incorporated by reference. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word comprise and variations of the word, such as comprising and comprises, means including but not limited to, and is not intended to exclude, for example, other additives, components, integers or steps.

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### EXAMPLES

### Example 1

### HIV-1 RT mutants, V148I and Q151N, have Reduced dNTP Binding Affinity and HIV-1 Vectors Containing these dNTP Binding Mutant RTs Fail to Transduce Cells Containing Low dNTP Concentrations

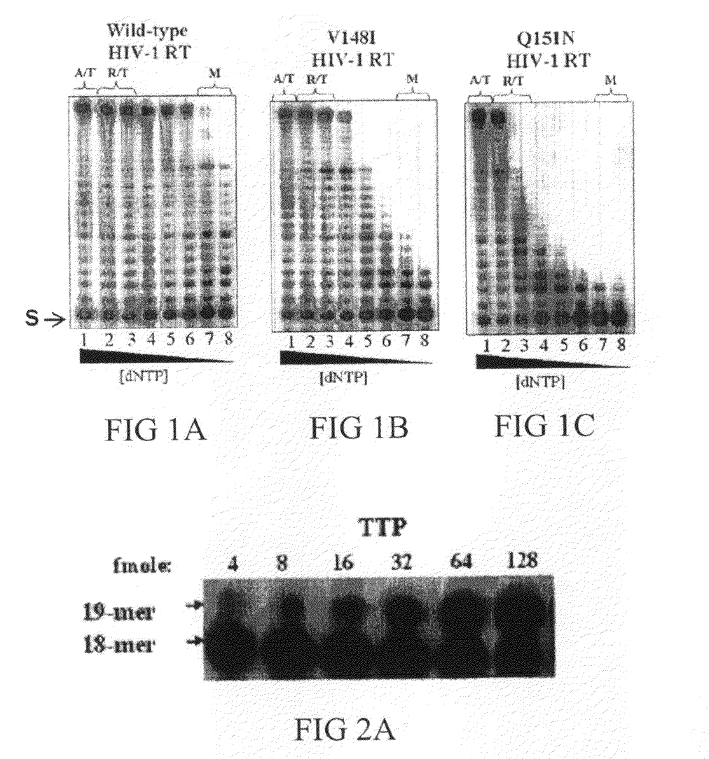

Pre-steady kinetic analysis of the HIV-1 RT Q151N and V148I mutants revealed that these RT mutations reduce the dNTP binding affinity of RT (increased Kd) without altering catalysis (kpol) (Diamond et al., J. Biol. Chem. 278:29913-24 (2003), Jamburuthugoda, Biochemistry 44:10635-43 (2005), Weiss et al., J. Biol. Chem. 277:22662-9 (2002)). Due to the reduced dNTP binding affinity, these RT mutants showed a severe reduction of DNA synthesis (primer extension) at low dNTP concentrations (lanes 7 and 8 of FIGS. 1B and 1C), in contrast to wild type HIV-1 RT (lanes 7 and 8 of FIG. 1A). However, these RT mutants still show wild type activity at high dNTP concentrations (i.e. 5 and 2.5 μM, lanes 1 and 2 of FIG. 1). This finding shows that, unlike wild type HIV-1 RT, the dNTP binding step of the Q151N and V148I mutants become rate limiting in DNA synthesis at low dNTP concentrations, due to the greatly reduced dNTP binding affinity of these RT mutants. Structural analysis demonstrated that the V148I and Q151N mutations interfere with the formation of the H-bond between the side chain of the Q151 residue and the 3′ OH of the incoming dNTP.

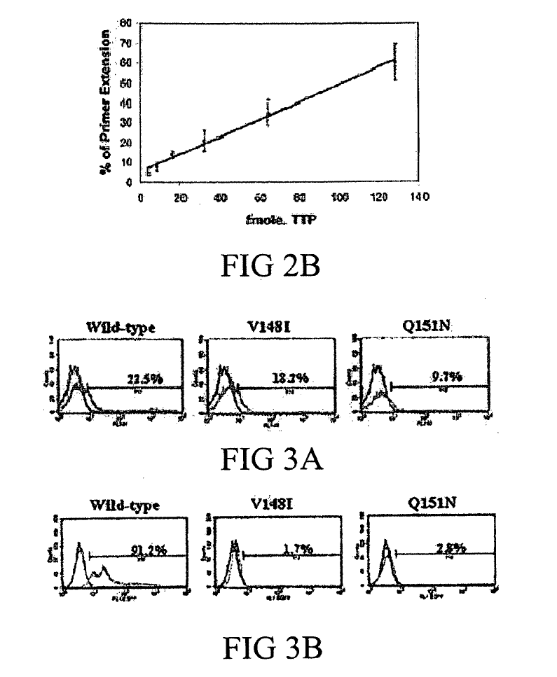

A highly sensitive dNTP assay capable of monitoring dNTP levels in primary cells has been described (Diamond et al., J. Biol. Chem. 279:51545-53 2004). It has been difficult to determine dNTP levels in terminally differentiated primary cells due to the lack of highly sensitive dNTP assays. A dNTP assay has been developed that can detect 4 fmole of dNTP and shows a linearity between 4128fmole (FIG. 2). This assay used a ³²P-labeled 18-mer primer annealed to a 19-mer template, allowing incorporation of one of four dNTPs, generating 19-mer products (FIG. 2A). Indeed, this assay is ˜250 times more sensitive than the HPLC based assay. With this assay, dNTP concentrations in primary human macrophages, CD4+ T cells, and human fibroblasts were determined (Table 3). This assay also allowed monitoring of fluctuations of cellular dNTP contents, in different cell types, or under different phases of the cell cycle or culture conditions. Primary cells such as human primary lung fibroblast cells (HFL culture with 10% serum) have 5-10 lower dNTP content than tumor cells such as PANC1 cells (Table 4). Treatment with deoxynucleoside (dNs) enhanced cellular dNTP content in human lung fibroblasts (cultured with 10% serum) by 80 fold (Table 4).

In addition, dNTP concentrations of two pancreatic cancer cells, PANC1 and BxPC-3 and one lung cancer cell line, A549 were determined. These three human cell lines are epithelial cancer cells. As shown in Table 4, all three human cancer cell lines have very elevated dNTP concentrations, compared to normal human primary fibroblast cells.

TABLE 4
dNTP concentrations of human cells.

| Human cell types [dNTP] | μM |
|---|---|
| Monocyte derived macrophages | ~0.05 |
| Activated CD4+ T cell | 2~5 |
| Lung fibroblasts | 0.1~0.2 (1x) |
| Lung fibroblasts (+dN) | 20~90 (~80x) |
| PANC1 | 1.3~2.4 |
| BxPC-3 | 2.0~4.1 |
| 293T | 1~2.3 |
| A549 | 1.7~3.3 |
| CHME5 | 2.3~5.7 |

### Example 2

### HIV-1 Vectors Containing dNTP Binding Mutant RTs Transduce Target Cells in a dNTP Concentration-Dependent Manner

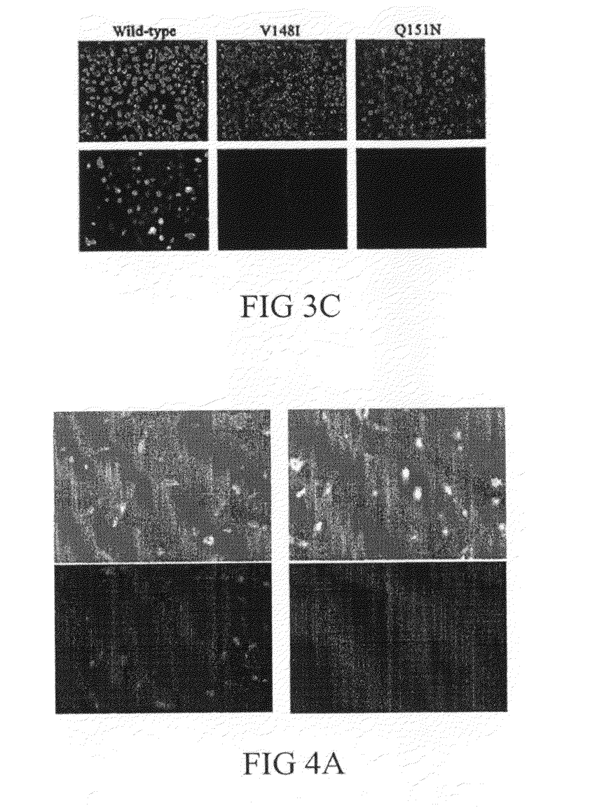

Using the dNTP assay described above, it was observed that human macrophages contain ˜50-100 fold lower cellular dNTP concentrations (˜50 nM) than actively dividing cells (CD4+ T cells, 2˜5 μM) and tumor cells (HeLa cells, 5˜10 μM) (see below). Since the V148I and Q155N dNTP binding mutants showed severely reduced DNA polymerization at the low concentrations found in macrophages (i.e., lane 7 and 8 in FIGS. 1B and 1C), dNTP binding HIV-1 RT mutations were tested for their ability to affect the transduction efficiency of a HIV-1 vector (expressing EGFP) in macrophages containing low cellular dNTP concentration. As shown in FIG. 3, a wild type HIV-1 vector can transduce both macrophages (FIGS. 3B and 3C) and cells containing high dNTP concentrations such as activated CD4+ T cells (FIG. 3A) and tumor cells (i.e. HeLa cells). In contrast, a HIV-1 vector containing the Q151N or V148I mutant RT failed to transducer macrophages (FIGS. 3B and 3C) even though these mutant viruses still efficiently transduce cells containing high dNTP concentrations (FIG. 3A). Therefore, these biochemical and virological findings suggest that the reduced dNTP binding affinity of HIV-1 RT is responsible for the failure of the HIV-1 vector to transduce cells containing low dNTP concentrations.

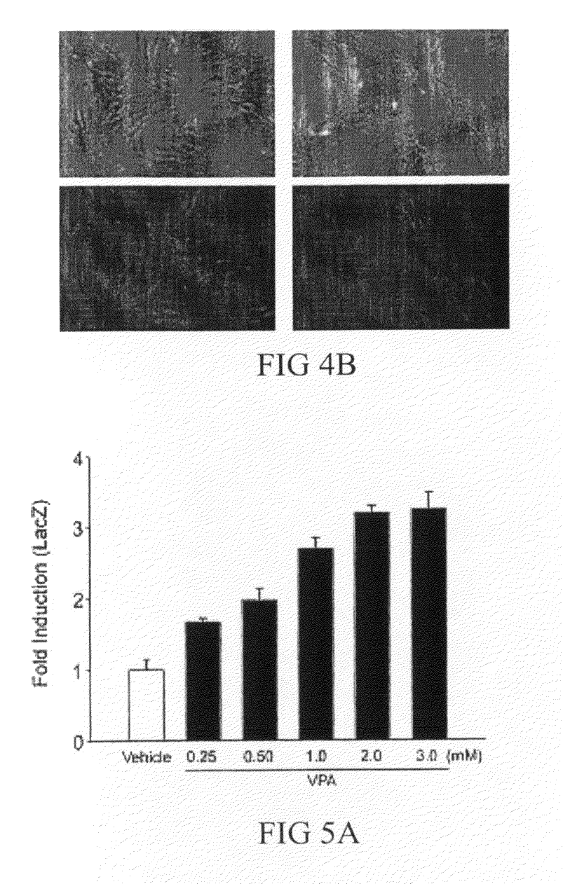

As shown in FIG. 1, the Q151N HIV-1 RT mutant showed significantly reduced primer extension even at 100-200 nM dNTP (lanes 5 and 6), whereas wild type HIV-1 RT still showed unchanged DNA polymerase activity in these dNTP concentrations. Since normal human primary fibroblast cells have ˜200 nM dNTP (Table 4), it was determined whether the Q151N HIV-1 vector could transduce primary human fibroblast cells. As shown in FIG. 4A, unlike a wild type HIV-1 vector, which transduces human fibroblasts very efficiently, HIV-1 vectors containing the Q151N RT mutant have a greatly reduced capability to transduce human primary lung fibroblast cells (<1%). Since deoxynucleoside treatment of human fibroblast cells increased the cellular dNTP content to a level close to that found in cancer cell lines (Table 4), it was determined whether the Q151N HIV-1 vector could transduce human primary fibroblasts pretreated with deoxynucleosides (2.5 mM). Indeed, as shown in FIG. 4B, the Q151N mutant HIV-1 vector was able to transduce primary human lung fibroblasts pretreated with exogenous deoxynucleosides. Therefore, these data show that low cellular dNTP content is a limiting factor that restricts transduction by mutant HIV-1 vectors in primary human lung fibroblasts. Similar data were obtained with the V148I mutant virus.

### Example 3

### Mutation of Residue Y115 in the HIV-1 Reverse Transcriptase Results in a Reduction in Polymerase Activity at Low dNTP Concentrations

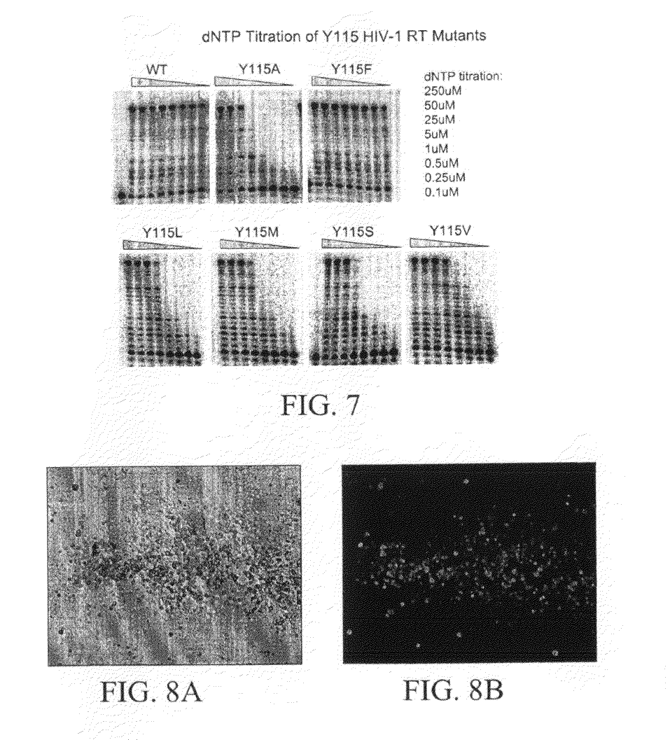

FIG. 7 shows a [dNTP]-dependent DNA polymerization assay with various HIV-1 RT polymerase mutants, and their wild-type counterpart (WT). A 5′ end ³²P end labeled DNA primer was annealed to a RNA template and extended by equivalent amounts of reverse transcriptase activity showing full extension of 50% labeled primers at 250 μM in a short reaction at 37° C. dNTP concentrations are denoted at the side of FIG. 7. The dark dots at the bottom of each lane represent the substrate, while the uppermost band in each panel represents the full-length extended product.

All of the Y115 mutants, with the exception of Y115F, are greatly impaired for DNA synthesis at low dNTP concentrations, but normal at high dNTP concentrations.

### Example 4

### An Adenovirus With I664V Polymerase Mutation Replicates in Cells

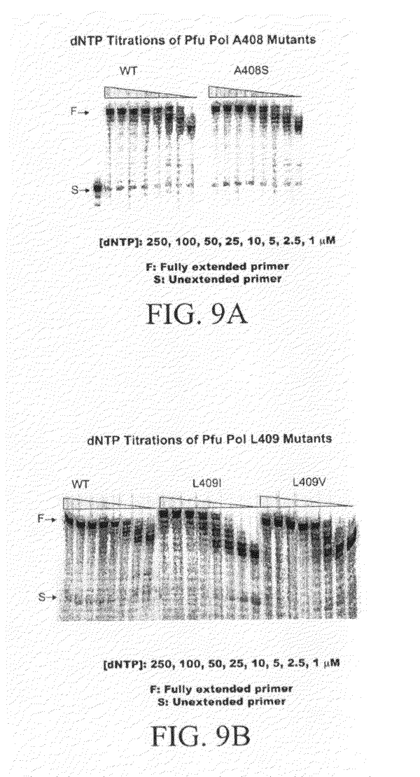

An adenovirus mutant containing the I664V polymerase mutation replicates in 293 cells in the presence of exogenous dNTPs (1 mM). FIG. 8 shows bright field (left) and fluorescence (right) images of a virus plaque in a 293 cell monolayer.

### Example 5

### Valproic Acid Enhances Adenovirally-Mediated Gene Expression in Vitro and In Vivo

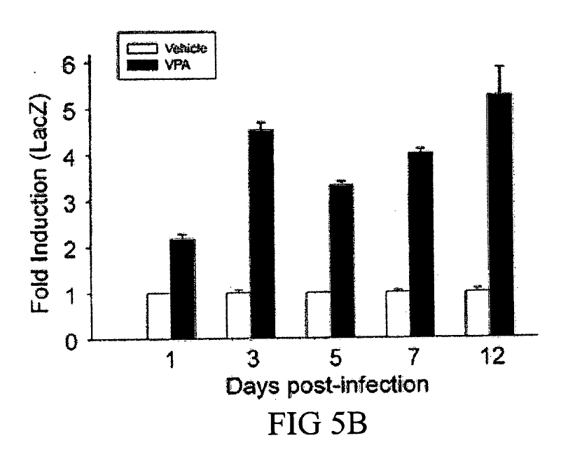

VPA enhanced virally-vectored gene expression, using a panel of gene delivery platforms that include recombinant, replication-defective type 5 adenovirus vectors. As shown in FIG. 5A, VPA treatment led to a dose-dependent increase in reporter gene expression following transduction of N2A neuroblastoma cells with an adenovirus vector expressing LacZ (Ad-CMV-LacZ). Similar effects were also observed in several primary cells transduced by Ad-CMV-LacZ (Fan, et al., J. Virol. Methods 125:23-33 (2005)). In addition, as shown in FIG. 5B, the increased expression of the delivered gene by VPA (1 mM) continued for more than 12 days post VPA-treatment.

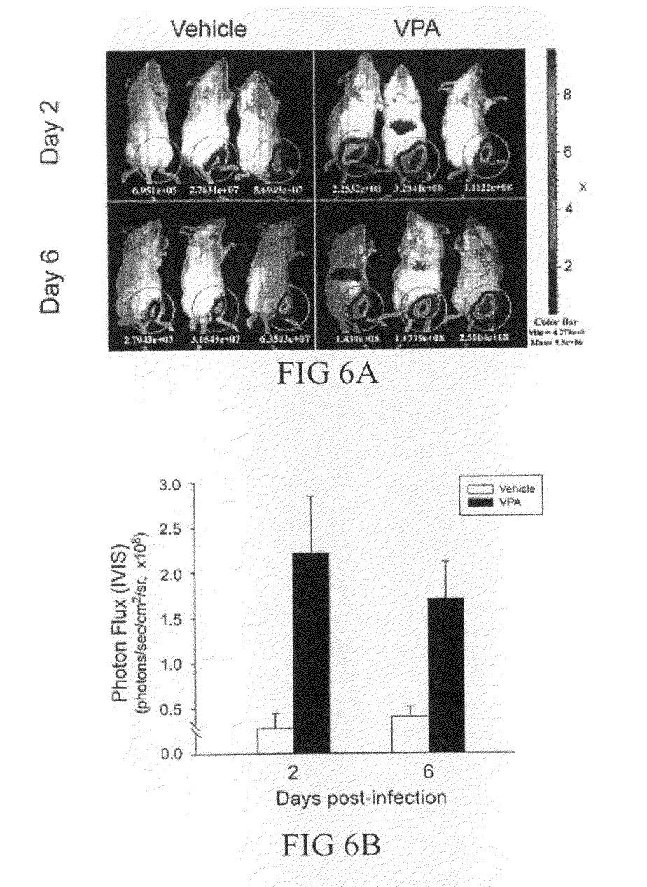

Next, it was determined that VPA treatment enhanced gene expression from a replication defective (E1A-deleted) Ad5 vector in vivo. 09 pfu of a luciferase-expressing adenovirus vector (Ad-CMV-Luc) was inoculated into BALB/c mice, via an intramuscular route. These animals were then treated with either VPA or with vehicle alone for six days, and the expression of luciferase was analyzed over time using biophotonic imaging. As shown in FIG. 6, the VPA treated mice showed increased expression of luciferase, compared to the untreated mice.

### Example 6

### Mutation of Residues 408, 409 and 410 in the Pfu DNA Polymerase Result in a Reduction in Polymerase Activity at Low dNTP Concentrations

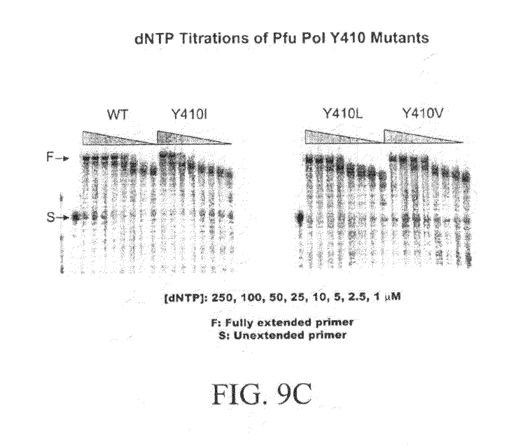

FIGS. 9A, 9B and 9C show [dNTP]-dependent DNA polymerization assays with various Pfu DNA polymerase mutants and their wild-type counterpart (WT). A 5′ end ³²P-end labeled DNA primer was annealed to a DNA template and extended by equivalent amounts of DNA polymerase activity showing full extension of 50% labeled primers at 250 μM in a short reaction at 37° C. dNTP concentrations are denoted below each of FIGS. 9A, 9B and 9C. S denotes the substrate, F denotes the full-length extended product. The L409I, L409V, Y410I, Y410L and Y410V mutants are impaired for DNA synthesis at low dNTP concentrations, but normal at high dNTP concentrations.

A number of embodiments of the invention have been described. Nevertheless, it will be understood that various modifications may be made without departing from the spirit and scope of the invention. Accordingly, other embodiments are within the scope of the following claims.

1. A viral vector comprising a nucleic acid encoding a viral DNA polymerase, wherein the encoded viral DNA polymerase comprises at least one amino acid mutation.

2. The viral vector of claim 1, wherein the viral vector is selected from the group consisting of adenoviral vectors, herpes simplex virus type-1 vectors, herpes simplex virus type-2 vectors, poxvirus vectors, lentiviral vectors, spumavirus vectors, and cytomegalovirus vectors.

3. (canceled)

4. The viral vector of claim 2, wherein the adenoviral vector is selected from the group consisting of an adenovirus type 3, adenovirus type 5, adenovirus type 7, adenovirus type 11, adenovirus type 19, adenovirus type 35, adenovirus type 49 and a non-human adenovirus vector.

5. The viral vector of claim 1, wherein the DNA polymerase is selected from the group consisting of an adenoviral DNA polymerase, a herpes simplex virus type-1 DNA polymerase, a herpes simplex virus type-2 DNA polymerase, a poxvirus DNA polymerase, a lentiviral DNA polymerase, a spumavirus DNA polymerase, and a cytomegalovirus DNA polymerase.

6. The viral vector of claim 5, wherein the adenoviral DNA polymerase is adenovirus type 5 DNA polymerase or an adenovirus type 19 DNA polymerase.

7. The viral vector of claim 6, wherein the mutation is an amino acid substitution selected from the group consisting of I664S, I664M, R665K, R833K, R833T, N841Y, N841S, Y844S, S692A, Y1010F, I664V, GG666/7AA, Y690A, Y690F and K837A.

8. The viral vector of claim 5, wherein the human lentiviral DNA polymerase is HIV-1 reverse transcriptase.

9. The viral vector of claim 8, wherein the mutation is an amino acid substitution selected from the group consisting of Q151N, V148I, A114V, A114L, A114S, Y115L, Y115M, Y115S, Y115V and Y115A.

10. The viral vector of claim 5, wherein the simian lentivirus DNA polymerase is simian immunodeficiency virus reverse transcriptase.

11. The viral vector of claim 10, wherein the mutation is amino acid substitution V148I.

12. The viral vector of claim 1, wherein the vector further comprises an essential viral gene operably linked to a tumor specific transcriptional promoter.

13. The viral vector of claim 1, wherein the vector further comprises a gene that functionally complements an essential viral gene and wherein the essential viral gene is non-functional.

14. The viral vector of claim 1, wherein the DNA polymerase is active at high dNTP concentrations.

15. The viral vector of claim 1, wherein the DNA polymerase is active at dNTP concentrations about 5 to 200 times higher than normal cellular dNTP concentrations.

16. The viral vector of claim 1, wherein the DNA polymerase shows 10 to 1000 fold lower activity at normal cellular dNTP concentrations as compared to its activity at high dNTP concentrations.

17. The viral vector of claim 1, wherein the vector further comprises a nucleic acid encoding a therapeutic polypeptide.

18. A pharmaceutical composition comprising the viral vector of claim 1 and a pharmaceutically acceptable carrier.

19. (canceled)

20. A method of treating cancer in a subject comprising administering to the subject the composition of claim 18.

21. The method of claim 20, further comprising administration of valproic acid or other histone deacetylase inhibitor (HDACi).

22. (canceled)

23. The method of claim 20, further comprising administration of radiation.

24. (canceled)

25. (canceled)


SEQUENCE LISTING (9 sequences)

SEQ ID NO: 1 - protein, 4 aa, Artificial Sequence
  Exemplary motif
XXGG

SEQ ID NO: 2 - protein, 4 aa, Artificial Sequence
  Exemplary motif
IXGG

SEQ ID NO: 3 - protein, 33 aa, Artificial Sequence
  Synthetically generated peptide
PVQPYPGAFVKEPIPNRYKYVMSFDLTSLYPSI

SEQ ID NO: 4 - protein, 33 aa, Artificial Sequence
  Synthetically generated peptide
KRESYTGGFVKEPEKGLWENIVYLDFRALYPSI

SEQ ID NO: 5 - protein, 36 aa, Artificial Sequence
  Synthetically generated peptide
VRYAYRGGFTWLNDRFKEKEIGEGMVFDVNSLYPAQ

SEQ ID NO: 6 - protein, 34 aa, Artificial Sequence
  Synthetically generated peptide
VRASIRGGRCYPTYLGILREPLYVYDICGMYASA

SEQ ID NO: 7 - protein, 43 aa, Artificial Sequence
  Synthetically generated peptide
EITKVFNQRKEHKGYMLARTEVAGMTAQINRKLLINSLYGALG

SEQ ID NO: 8 - protein, 36 aa, Artificial Sequence
  Synthetically generated peptide
LFKDFIDKWTYIKTTSEGAIKQLAKLMLNSLYGKFA

SEQ ID NO: 9 - protein, 39 aa, Artificial Sequence
  Synthetically generated peptide
VAREYVQLNIAAKEADRDKNQLTRSIAKLLSNALYGSFA